(19) United States
(12) Patent Application Publication
(10) Pub. No.: US 2025/0230361 A1
(43) Pub. Date: Jul. 17, 2025

(54) USES, METHODS AND COMPOSITIONS

(71) Applicant: INNOSPEC ACTIVE CHEMICALS LLC, High Point, NC (US)

(72) Inventors: Susan PYE, Ellesmere Port (GB); Peter CLARK, Ellesmere Port (GB); Stephen Moss O'CONNOR, High Point, NC (US); Russell COX, Ellesmere Port (GB)

(21) Appl. No.: 19/016,602

(22) Filed: Jan. 10, 2025

Related U.S. Application Data
(60) Provisional application No. 63/620,277, filed on Jan. 12, 2024.

(30) Foreign Application Priority Data
Feb. 12, 2024 (GB) 2401886.3

(51) Int. Cl. C09K 23/28; C09K 23/00; C09K 23/04; C09K 23/16

(52) U.S. Cl. CPC C09K 23/28; C09K 23/018; C09K 23/04; C09K 23/16

(57) ABSTRACT

Use of a chelating agent in a surfactant composition to provide transparency and/or to substantially prevent discolouration, wherein the chelating agent comprises at least one aminopolycarboxylic acid or a salt thereof.

### FIELD

The present invention relates to surfactant compositions that do not substantially discolour over time (such as when stored) and which may be transparent. The present invention also relates to the use of chelating agents to provide transparency and/or to substantially prevent discolouration in a surfactant composition.

### BACKGROUND

Surfactants are widely used in many industries and products, including personal care, household and industrial products. It is desirable, particularly for consumers, that products comprising surfactants have an appealing appearance. However in some cases consumers have noted that surfactants, and compositions comprising surfactants, may have issues with instability (for example upon storage) which can cause discolouration. Typically, a yellow discolouration occurs. Such discolouration can be undesirable to consumers, as well as to manufacturers using the surfactant or surfactant composition. It is therefore desirable for surfactants and surfactant compositions to maintain their stability upon storage without discolouring. In some cases, it is also desirable to provide transparent surfactant compositions.

It is thus an object of the present invention to provide a surfactant composition that does not substantially discolour upon storage. It is another object of the invention to provide a transparent surfactant composition, for example that does not substantially discolour upon storage.

### SUMMARY OF THE INVENTION

According to aspects of the present invention, there is provided a use, method and composition as set forth in the appended claims. Other features of the invention will be apparent from the dependent claims, and from the description which follows.

The inventors have identified that discolouration of a surfactant composition may occur due to the degradation (for example upon storage) of one or more additives, such as chelating agents, that are contained in the composition. For example, some chelating agents, such as phosphonate-based chelating agents, may degrade upon storage and thereby discolour, typically leading to discolouration of the surfactant composition containing the chelating agent. However the inventors have found that chelating agents comprising at least one aminopolycarboxylic acid or a salt thereof do not cause discolouration of a surfactant composition.

According to a first aspect of the present invention, there is provided a use of a chelating agent in a surfactant composition to provide transparency and/or to substantially prevent discolouration, wherein the chelating agent comprises at least one aminopolycarboxylic acid or a salt thereof.

According to a second aspect of the present invention, there is provided a method of providing transparency to and/or substantially preventing discolouration of a surfactant composition, the method comprising adding a chelating agent to the surfactant composition, wherein the chelating agent comprises at least one aminopolycarboxylic acid or a salt thereof.

According to a third aspect of the present invention, there is provided a transparent surfactant composition comprising:
(i) from 15 to 98 wt % of at least one acyl alkyl isethionate surfactant of the formula (I):

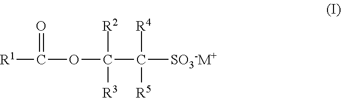

wherein R¹ represents an optionally substituted C₃-C₃₅ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
(ii) from 3 to 12 wt % of at least one amphoteric surfactant; and
(iii) from 0.1 to 10 wt % of a chelating agent, wherein the chelating agent comprises at least one aminopolycarboxylic acid or a salt thereof.

According to the fourth aspect of the present invention, there is provided a personal or household cleaning composition comprising a transparent surfactant composition according to the third aspect.

According to a fifth aspect of the present invention, there is provided a method of preparing a transparent surfactant composition according to the third aspect, the method comprising admixing the at least one acyl alkyl isethionate of the formula (I), the at least one betaine surfactant and the at least one chelating agent in the amounts set out in the third aspect.

Other features of the invention will be apparent from the dependent claims, and from the description which follows.

Features described in relation to the second, third, fourth and fifth aspects may have any of the suitable features and advantages described in relation to the first aspect.

### DETAILED DESCRIPTION OF THE INVENTION

Unless otherwise stated, the following terms used in the specification and claims have the meanings set out below.

As used herein, the term “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include hydrocarbon groups, i.e. aliphatic (which may be saturated or unsaturated, linear or branched, for example alkyl or alkenyl), alicyclic (for example cycloalkyl, cycloalkenyl) and aromatic (for example phenyl) groups.

The term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “C₃-C₃₅ alkyl” includes C₁₀-C₃₅ alkyl, C₄-C₆ alkyl, propyl, isopropyl and t-butyl.

The term “alkenyl” includes both straight and branched chain alkenyl groups. References to individual alkenyl groups such as “propenyl” are specific for the straight chain version only and references to individual branched chain alkenyl groups such as “isopropenyl” are specific for the branched chain version only. For example, “C₃-C₃₅ alkenyl” includes C₁₀-C₃₅ alkenyl, C₄-C₆ alkenyl, propenyl and isopropenyl.

The term “alkoxy” includes both straight and branched chain alkoxy groups. References to individual alkoxy groups such as “propoxy” are specific for the straight chain version only and references to individual branched chain alkyl groups such as “isopropoxy” are specific for the branched chain version only. For example, “C₁-C₄ alkoxy” includes C₁-C₂ alkoxy, propoxy, isopropoxy and t-butoxy.

The term “aryl” means a cyclic or polycyclic aromatic ring having from 5 to 12 carbon atoms. Examples of aryl groups include, but are not limited to, phenyl, biphenyl and naphthyl. In a particular embodiment, an aryl group may be phenyl.

The term “C₈-C₂₂ alkyl-C₆-C₁₂ aryl” means a C₈-C₁₂ aryl group covalently attached to a C₈-C₂₂ alkyl group, both of which are defined herein.

The term “C₆-C₁₂ aryl-C₈-C₂₂ alkyl” means a C₈-C₂₂ alkyl group covalently attached to a C₆-C₁₂ aryl group, both of which are defined herein.

The term “optionally substituted” with reference to a particular group, such as a hydrocarbyl group, alkyl group, alkenyl group, alkoxy group, or aryl group means that said group may be substituted or unsubstituted. Suitable substituents may include non-hydrocarbon groups provided that they do not alter the predominantly hydrocarbon nature of the group. Examples of suitable substituents include C₁₄ alkoxy, cyano, hydroxy, oxo, halo (especially fluoro and chloro), trifluoromethyl and trifluoromethoxy.

Unless stated to be optionally substituted, the hydrocarbyl groups, alkyl groups, alkenyl groups, alkoxy groups, and aryl groups herein are unsubstituted.

According to a first aspect of the present invention, there is provided a use of a chelating agent in a surfactant composition to provide transparency and/or to substantially prevent discolouration, wherein the chelating agent comprises at least one aminopolycarboxylic acid or a salt thereof.

The use of the first aspect therefore provides transparency to the surfactant composition and/or substantially prevents discolouration of the surfactant composition. In other words, in the use of the first aspect, the chelating agent acts to make the surfactant composition transparent and/or to substantially prevent discolouration of the surfactant composition (i.e. to which the chelating agent is added). The surfactant composition may not be transparent, for example the surfactant composition may be opaque, in the absence of the chelating agent.

The first aspect of the present invention may provide a use of a chelating agent in a surfactant composition to provide transparency and to substantially prevent discolouration, wherein the chelating agent comprises at least one aminopolycarboxylic acid or a salt thereof.

The first aspect of the present invention may provide a use of a chelating agent in a surfactant composition to provide transparency, wherein the chelating agent comprises at least one aminopolycarboxylic acid or a salt thereof.

The first aspect of the present invention may provide a use of a chelating agent in a surfactant composition to substantially prevent discolouration, wherein the chelating agent comprises at least one aminopolycarboxylic acid or a salt thereof.

According to a second aspect of the present invention, there is provided a method of providing transparency to and/or substantially preventing discolouration of a surfactant composition, the method comprising adding a chelating agent to the surfactant composition, wherein the chelating agent comprises at least one aminopolycarboxylic acid or a salt thereof.

According to a second aspect of the present invention, there is provided a method of providing transparency to and substantially preventing discolouration of a surfactant composition, the method comprising adding a chelating agent to the surfactant composition, wherein the chelating agent comprises at least one aminopolycarboxylic acid or a salt thereof.

According to a second aspect of the present invention, there is provided a method of providing transparency to a surfactant composition, the method comprising adding a chelating agent to the surfactant composition, wherein the chelating agent comprises at least one aminopolycarboxylic acid or a salt thereof.

According to a second aspect of the present invention, there is provided a method of substantially preventing discolouration of a surfactant composition, the method comprising adding a chelating agent to the surfactant composition, wherein the chelating agent comprises at least one aminopolycarboxylic acid or a salt thereof.

The use and method of the first and second aspects may act to provide a transparent surfactant composition. The use and method of the first and second aspects may act to provide a transparent surfactant composition of the third aspect of the present invention as described herein.

The use and method of the first and second aspects may act to substantially prevent discolouration of the surfactant composition. The use and method of the first and second aspects may act to completely prevent discolouration of the surfactant composition, i.e. such that no discolouration occurs. The use and method of the first and second aspects may act to provide a transparent surfactant composition and to substantially prevent discolouration of the surfactant composition. In other words, when the chelating agent is added to the surfactant composition, the resulting composition may be transparent and may not substantially discolour (or may not discolour completely), for example upon storage.

Thus, it has surprisingly been found that when a chelating agent comprising at least one aminopolycarboxylic acid or a salt thereof is added to a surfactant composition it may cause the composition to become transparent. It has also surprisingly been found that when a chelating agent comprising at least one aminopolycarboxylic acid or a salt thereof is added to a surfactant composition it may prevent any substantial discolouration of the composition or may completely prevent discolouration of the surfactant composition.

Furthermore, it has surprisingly been found that when a chelating agent comprising at least one aminopolycarboxylic acid or a salt thereof is added to a surfactant composition it may cause the composition to become transparent such that the transparency is maintained over time without any substantial discolouration (or without any discolouration) of the composition. In contrast, other chelating agents may fail to prevent discolouration over time. For example, other chelating agents may provide transparency but fail to prevent discolouration over time. For example, use of phosphonate- or phosphonic acid-derived chelating agents has been found to result in discolouration in surfactant compositions. Phosphonate- or phosphonic acid-derived chelating agents include aminotri(methylenephosphonic acid) (ATMP), ethylenediamine tetra(methylenephosphonic acid) (EDTMP), 1-hydroxylethylidene-1,1-diphosphonic acid (HEDP) and diethylenetriaminepentamethylene phosphonic acid (DTPMPA) and salts or partial salts thereof.

Typically, addition of a chelating agent comprising at least one aminopolycarboxylic acid or a salt thereof to a surfactant composition provides transparency, which is retained and wherein discolouration is substantially prevented over a period of time, such as upon storage of the surfactant composition (i.e. after addition of the chelating agent). The discolouration may be completely prevented, i.e. such that no discolouration occurs.

The term “transparent” (or the term “transparency”) would be well understood by a person skilled in the art to mean that light can pass through the material or composition, such that an object behind can be distinctly seen.

As a person skilled in the art would appreciate, transparency can be measured in different ways, such as by measuring the amount of light that passes through a sample, so as to provide a transmittance value (% T), or by measuring the scattering of light by a sample, so as to provide an indication of turbidity (determined by Nephelometric Turbidity Units or NTU). Thus, transmittance and turbidity values are both properties that can be used to indicate whether or not a sample is transparent. The selection of which method to use to determine whether a sample is transparent will depend on the nature of the sample, such as the components thereof and whether the sample comprises any suspended solids, as would be understood by a person skilled in the art.

A suitable transmittance test can, for example, be conducted using a Photometer 7100 instrument (for example available from Palintest), which records a transmittance value as a percentage of visible light transmitted through a sample, for example as measured according to the transmittance test measurement method described in the examples. An opaque sample will have a transmittance value of 0%. Herein a composition having a transmittance value (% T) of 90% or greater is considered to be transparent.

A suitable test for turbidity may be conducted using a HunterLab Vista instrument, for example as measured according to the turbidity value test method described in the examples. Herein a composition having a turbidity value of less than 10 NTU is considered to be transparent.

Thus, herein, a composition having a transmittance value (% T) of 90% or greater and/or a turbidity value of less than 10 NTU is considered to be transparent. Suitably herein, a transparent composition may have a transmittance value (% T) of 90% or greater and a turbidity value of less than 10 NTU.

Suitably, a surfactant composition comprising a chelating agent comprising at least one aminopolycarboxylic acid or a salt thereof (such as a composition according to the third aspect of the invention) may have a transmittance value of 90% or greater, for example 95% or greater, such as 98% or greater, most preferably 100% (for example as measured according to the transmittance test measurement method described in the examples).

Suitably, a surfactant composition comprising a chelating agent comprising at least one aminopolycarboxylic acid or a salt thereof (such as a composition according to the third aspect of the invention) may have a turbidity value of less than 10 NTU, suitably less than 5 NTU.

Suitably, a surfactant composition comprising a chelating agent comprising at least one aminopolycarboxylic acid or a salt thereof (such as a composition according to the third aspect of the invention) may have a transmittance value of 90% or greater, for example 95% or greater, such as 98% or greater, most preferably 100% (for example as measured according to the transmittance test measurement method described in the examples) and/or a turbidity value of less than 10 NTU, suitably less than 5 NTU.

For example, a surfactant composition comprising a chelating agent comprising at least one aminopolycarboxylic acid or a salt thereof (such as a composition according to the third aspect of the invention) may have a transmittance value of 90% or greater, for example 95% or greater, such as 98% or greater, most preferably 100% (for example as measured according to the transmittance test measurement method described in the examples) and a turbidity value of less than 10 NTU, suitably less than 5 NTU.

Thus, by “providing or to provide transparency” we mean that addition of the chelating agent to a surfactant composition provides a transparent surfactant composition (i.e. comprising the chelating agent in addition to one or more surfactants) as measured by the transmittance value (% T) and/or as measured by the turbidity value.

Suitably, the surfactant composition prior to addition of the chelating agent as defined herein is not transparent, such that the surfactant composition has a transmittance value (% T) of less than 90% and/or a turbidity value of 10 or greater. For example the surfactant composition prior to addition of the chelating agent may be opaque, i.e. have a transmittance value (% T) of 0%.

By “discolouration” we mean unintended colouration. Typically, the discolouration may be a yellow discolouration, i.e. a yellowing of a sample or of the surfactant composition.

Discolouration by yellowing may be measured by means of a yellowness index test. The skilled person would be familiar with such a test. For example, the yellowness index may be measured using a Lovibond® Comparator 2000+ instrument using the standard method for the instrument (for example according to method 1 disclosed in the examples) or using a HunterLab Vista Spectrophotometer instrument according to ASTM D5386 (for example according to method 2 disclosed in the examples). These tests record a yellowness index in Hazen or APHA units.

By “substantially preventing discolouration” we mean that any discolouration that does occur is minimal, for example, wherein the yellowness index of a sample or the surfactant composition is 125 Hazen units or less, preferably 80 Hazen units or less, for example upon storage for at least 4 weeks (such as at least 4 weeks at 25° C. and upon exposure to light). By “completely preventing discolouration”, we mean that no (i.e. zero) discolouration occurs.

The transparent surfactant composition herein may be coloured or colourless, preferably colourless. By colourless we mean that the composition lacks substantial colour to the naked human eye when viewed in white light with a white background. Lack of colour can be measured by means of the yellowness test as disclosed herein. For example, a yellowness index of 125 Hazen units or less may be considered colourless. 1 Hazen unit is equivalent to the colour produced by 1 milligram of platinum (present as hexachloroplatinate (IV) in association with cobalt (II) chloride) per litre of water.

Typically, the discolouration process may be accelerated by storage upon exposure to light (such as sunlight) and/or at temperatures above ambient temperature. Accelerated stability testing methods, for example conducted at temperatures above ambient temperature, may be used to provide an indication of long-term stability at ambient temperature.

The chelating agent comprising at least one aminopolycarboxylic acid or a salt thereof may provide transparency and/or substantially prevent discolouration upon storage for a period of time, such as a period of 1 to 2 years, or a period of 1 to 3 years, at ambient temperature (with or without exposure to light).

The chelating agent comprising at least one aminopolycarboxylic acid or a salt thereof may provide transparency and substantially prevent discolouration upon storage for a period of time, such as a period of 1 to 2 years, or a period of 1 to 3 years, at ambient temperature (with or without exposure to light).

The chelating agent comprising at least one aminopolycarboxylic acid or a salt thereof may provide transparency and/or substantially prevent discolouration upon storage for a period of time, such as a period of at least 2 weeks, for example at least 4, 6 or 8 weeks, or at least 4, 6, 8 or 12 weeks, at a temperature above ambient temperature, such as a temperature of 40 to 50° C., for example about 45° C. (with or without exposure to light).

The chelating agent comprising at least one aminopolycarboxylic acid or a salt thereof may provide transparency and/or substantially prevent discolouration upon storage for a period of time, such as a period of at least 2 weeks, for example at least 4, 6 or 8 weeks, or at least 4, 6, 8 or 12 weeks, at a temperature below ambient temperature, such as a temperature of 0 to 10° C., for example about 5° C. (with or without exposure to light).

The chelating agent comprising at least one aminopolycarboxylic acid or a salt thereof may provide transparency and/or may substantially prevent discolouration upon storage for a period of at least 2 weeks (for example at least 4, 6 or 8 weeks, or at least 4, 6, 8 or 12 weeks), for example at a temperature above ambient temperature and/or upon exposure to light.

The chelating agent comprising at least one aminopolycarboxylic acid or a salt thereof may provide transparency and/or may substantially prevent discolouration upon storage for a period of at least 2 weeks (for example at least 4, 6 or 8 weeks, or at least 4, 6, 8 or 12 weeks), for example at a temperature below ambient temperature and/or upon exposure to light.

The chelating agent comprising at least one aminopolycarboxylic acid or a salt thereof may provide a transparent surfactant composition having a transmittance value of 90% or greater and/or a yellowness index of 125 Hazen units or less (such as 80 Hazen units or less) upon storage for a period of at least 2 weeks (for example at least 4, 6 or 8 weeks, or at least 4, 6, 8 or 12 weeks), for example at a temperature above ambient temperature and/or upon exposure to light.

The chelating agent comprising at least one aminopolycarboxylic acid or a salt thereof may provide a transparent surfactant composition having a transmittance value of 90% or greater and/or a yellowness index of 125 Hazen units or less (such as 80 Hazen units or less) upon storage for a period of at least 2 weeks (for example at least 4, 6 or 8 weeks, or at least 4, 6, 8 or 12 weeks), for example at a temperature below ambient temperature and/or upon exposure to light.

The chelating agent comprising at least one aminopolycarboxylic acid or a salt thereof may provide transparency and/or may substantially prevent discolouration upon storage for a period of at least 2 weeks (for example at least 4, 6 or 8 weeks, or at least 4, 6, 8 or 12 weeks) at a temperature above ambient temperature and upon exposure to light.

The chelating agent comprising at least one aminopolycarboxylic acid or a salt thereof may provide a transparent surfactant composition having a transmittance value of 90% or greater and/or a yellowness index of 125 Hazen units or less (such as 80 Hazen units or less) upon storage for a period of at least 2 weeks (for example at least 4, 6 or 8 weeks, or at least 4, 6, 8 or 12 weeks) at a temperature above ambient temperature and upon exposure to light.

The chelating agent comprising at least one aminopolycarboxylic acid or a salt thereof may provide a transparent surfactant composition without substantial discolouration upon storage for a period of at least 2 weeks (for example at least 4, 6 or 8 weeks, or at least 4, 6, 8 or 12 weeks) at a temperature above ambient temperature (with or without exposure to light).

The chelating agent comprising at least one aminopolycarboxylic acid or a salt thereof may provide a transparent surfactant composition having a transmittance value of 90% or greater and/a yellowness index of 125 Hazen units or less (such as 80 Hazen units or less) upon storage for a period of at least 2 weeks (for example at least 4, 6 or 8 weeks, or at least 4, 6, 8 or 12 weeks) at a temperature above ambient temperature (with or without exposure to light).

The chelating agent comprising at least one aminopolycarboxylic acid or a salt thereof may provide transparency and/or may substantially prevent discolouration upon storage for a period of at least 2 weeks (for example at least 4, 6 or 8 weeks, or at least 4, 6, 8 or 12 weeks) at a temperature below ambient temperature and upon exposure to light.

The chelating agent comprising at least one aminopolycarboxylic acid or a salt thereof may provide a transparent surfactant composition having a transmittance value of 90% or greater and/or a yellowness index of 125 Hazen units or less (such as 80 Hazen units or less) upon storage for a period of at least 2 weeks (for example at least 4, 6 or 8 weeks, or at least 4, 6, 8 or 12 weeks) at a temperature above below temperature and upon exposure to light.

The chelating agent comprising at least one aminopolycarboxylic acid or a salt thereof may provide a transparent surfactant composition without substantial discolouration upon storage for a period of at least 2 weeks (for example at least 4, 6 or 8 weeks, or at least 4, 6, 8 or 12 weeks) at a temperature below ambient temperature (with or without exposure to light).

The chelating agent comprising at least one aminopolycarboxylic acid or a salt thereof may provide a transparent surfactant composition having a transmittance value of 90% or greater and/a yellowness index of 125 Hazen units or less (such as 80 Hazen units or less) upon storage for a period of at least 2 weeks (for example at least 4, 6 or 8 weeks, or at least 4, 6, 8 or 12 weeks) at a temperature below ambient temperature (with or without exposure to light).

The chelating agent comprising at least one aminopolycarboxylic acid or a salt thereof may provide a transparent surfactant composition without substantial discolouration upon storage for a period of 1 to 2 years, or a period of 1 to 3 years, at ambient temperature (with or without exposure to light).

The chelating agent comprising at least one aminopolycarboxylic acid or a salt thereof may provide a transparent surfactant composition having a transmittance value of 90% or greater and a yellowness index of 125 Hazen units or less (such as 80 Hazen units or less) upon storage for a period of 1 to 2 years, or a period of 1 to 3 years, at ambient temperature (with or without exposure to light).

The chelating agent comprising at least one aminopolycarboxylic acid or a salt thereof may provide a transparent surfactant composition without substantial discolouration upon storage for a period of at least 2 weeks (for example at least 4, 6 or 8 weeks, or at least 4, 6, 8 or 12 weeks) upon exposure to light.

The chelating agent comprising at least one aminopolycarboxylic acid or a salt thereof may provide a transparent surfactant composition having a transmittance value of 90% or greater and a yellowness index of 125 Hazen units or less (such as 80 Hazen units or less) upon storage for a period of at least 2 weeks (for example at least 4, 6 or 8 weeks, or at least 4, 6, 8 or 12 weeks) upon exposure to light.

References herein to “exposure to light” may include continual or intermittent light exposure.

By ambient temperature, we mean a temperature of about 25° C. By temperatures above ambient temperature, we mean a temperature above 25° C., for example a temperature of from 26 to 60° C. (such as from 40 to 50° C., for example about 45° C.). By temperatures below ambient temperature, we mean a temperature below 25° C., for example a temperature of from 24 to 0° C. (such as from 0 to 10° C., for example about 5° C.).

The skilled person will recognise that storage at temperatures above ambient temperature (such as at temperatures of 40 to 50° C., for example about 45° C.) and/or exposure to light will cause accelerated degradation. Storage under such conditions may provide an indication of stability when stored under ordinary conditions, such as storage in a sealed container at ambient temperature. If a composition is stable after 8 weeks of accelerated testing it is expected to be stable for at least 2 years under ordinary conditions. If a composition is stable after 12 weeks of accelerated testing it is expected to be stable for at least 3 years under ordinary conditions.

The chelating agent comprising at least one aminopolycarboxylic acid or a salt thereof may provide a transparent surfactant composition without substantial discolouration upon storage in a sealed container at ambient temperature and without exposure to light for a period of at least 8 weeks, or at least 12 weeks, such as at least 1 year, preferably at least 2 years, or at least 3 years.

The chelating agent comprising at least one aminopolycarboxylic acid or a salt thereof may provide a transparent surfactant composition having a transmittance value of 90% or greater and/or a yellowness index of 12 Hazen units or less (such as 80 Hazen units or less) upon storage for a period of at least 1 year, preferably at least 2 years, or at least 3 years, in a sealed container at ambient temperature and without exposure to light.

The chelating agent comprising at least one aminopolycarboxylic acid or a salt thereof may provide a transparent surfactant composition (for example having a turbidity value of less than 10 NTU) without substantial discolouration upon storage in a sealed container at 5° C. and without exposure to light for a period of at least 8 weeks, suitably for period of at least 12 weeks.

The chelating agent comprising at least one aminopolycarboxylic acid or a salt thereof may provide a transparent surfactant composition (for example having a turbidity value of less than 10 NTU) without substantial discolouration upon storage in a sealed container at 25° C. and without exposure to light for a period of at least 8 weeks, suitably for period of at least 12 weeks.

The chelating agent comprising at least one aminopolycarboxylic acid or a salt thereof may provide a transparent surfactant composition (for example having a turbidity value of less than 10 NTU) without substantial discolouration upon storage in a sealed container at 45° C. and without exposure to light for a period of at least 8 weeks, suitably for period of at least 12 weeks.

In the first and second aspects, the surfactant composition to which the chelating agent comprising at least one aminopolycarboxylic acid or a salt thereof is added may be transparent prior to the addition of the chelating agent, but typically the surfactant composition becomes transparent after addition of the chelating agent.

In the first and second aspects, the chelating agent comprises at least one aminopolycarboxylic acid or a salt thereof. The chelating agent may comprise a mixture of two or more aminopolycarboxylic acids or salts thereof. By “aminopolycarboxylic acid or a salt thereof” we mean a molecule comprising two or more carboxylic acid moieties or salts thereof and one or more amine moieties. Aminopolycarboxylic acid salts herein include aminopolycarboxylic acids in which at least one carboxylic acid moiety is present as a salt. Any suitable aminopolycarboxylic acid or a salt thereof may be used as the chelating agent, as will be known to the person skilled in the art.

Suitably aminopolycarboxylic acid chelating agents for use herein may include 3, 4 or 5 carboxylic acid moieties. Suitably aminopolycarboxylic acid chelating agents for use herein may include 1, 2 or 3 amine moieties and these may be present as secondary or tertiary amines moieties. Preferably, the aminopolycarboxylic acid chelating groups do not comprise phosphonic acid groups or salts thereof.

The at least one aminopolycarboxylic acid may be selected from one or more of ethylenediamine-N,N′-diglutaric acid (EDDG), ethylenediamine-N,N′-dimalonic acid (EDDM), iminodisuccinic acid (IDS), ethylenediaminetetraacetic acid (EDTA), ethylenediaminedisuccinic acid (EDDS), methylglycinediacetic acid (MGDA), L-glutamic acid-N,N-diacetic acid (GLDA), diethylene triamine pentaacetic acid (DTPA), L-aspartic acid diacetic acid (ASDA), hydroxyethyl ethylenediaminetriacetic acid (HEDTA), iminodifumaric acid (IDF), iminoditartaric acid (IDT), iminodimaleic acid (IDMAL), iminodimalic acid (IDM), ethylenediaminedifumaric acid (EDDF), ethylenediaminedimalic acid (EDDM), ethylenediamineditartaric acid (EDDT) and ethylenediaminedimaleic acid (EDDMAL) or a salt thereof.

The at least one aminopolycarboxylic acid may be selected from one or more of ethylenediamine-N,N′-diglutaric acid (EDDG), ethylenediamine-N,N′-dimalonic acid (EDDM), iminodisuccinic acid (IDS), ethylenediaminetetraacetic acid (EDTA), ethylenediaminedisuccinic acid (EDDS), methylglycinediacetic acid (MGDA) and L-glutamic acid-N,N-diacetic acid (GLDA), preferably from EDTA, EDDS, MGDA and GLDA, or a salt thereof.

Suitable salts of the aminopolycarboxylic acid include amine salts, alkaline earth metal salts or alkali metal salts. Suitable amine salts include those of ammonia and triethanolamine. Suitable alkaline earth metal salts include those of magnesium and calcium. Suitable alkali metal salts include sodium, lithium and potassium salts. Preferably the chelating agent is a provided as a sodium salt. Suitable salts include partial salts where one or more but not all of the acid groups are present as salts.

EDTA has the structure shown in formula (A):

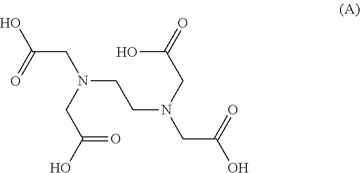 [SMILES: O=C(O)CN(CCN(CC(=O)O)CC(=O)O)CC(=O)O]

When the chelating agent comprises EDTA, this may be provided in a form having the structure shown in formula (A) or in a form having the same structure in which a number of the hydrogen atoms have been replaced. Thus the EDTA chelating agent may comprise salts in which 1, 2, 3 or 4 of the acid groups have been neutralised or partially neutralised.

When a salt of EDTA is used, this may be the salt of an alkali metal, an alkaline earth metal, ammonia or a suitable amine.

When a monovalent counterion is used the salt may be the monosalt, the disalt, the trisalt or the tetrasalt. For a divalent cation the monosalt or disalt may be present. Mixed salts may also exist, for example, the disodium magnesium salt or the sodium magnesium salt may be present. Preferably the counterion(s) to the EDTA residue is/are selected from one or more of sodium, magnesium, calcium, potassium, lithium, ammonium, and a quaternary ammonium ion.

Preferably when the chelating agent is EDTA it is present as the tetrasodium salt.

Ethylenediamine disuccinic acid (EDDS) which has the structure shown in formula (B):

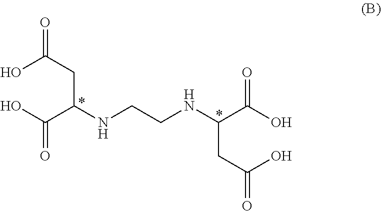 [SMILES: O=C(O)CC(NCCNC(CC(=O)O)C(=O)O)C(=O)O]

EDDS includes two stereogenic centres and there are three possible stereoisomers. A particularly preferred configuration is [S,S]-ethylenediamine disuccinic acid which is readily biodegradable.

EDDS may have the structure shown in formula (B) and/or the same structure in which a number of the hydrogen atoms have been replaced. Thus EDDS may also contain succinate salts in which 1, 2, 3 or 4 of the acid groups have been neutralised or partially neutralised. It may be present as a free acid or a salt or complex thereof.

One commercially available material is trisodium ethylenediamine disuccinate. The commercial product (Natrlquest E30 ®) is supplied as an aqueous solution comprising 30% by weight EDDS (expressed as free acid), or 37 wt % of the trisodium salt (including the counterion).

Another commercially available form of EDDS is the tetra acid, sold under the trade mark Natrlquest E80. This is provided as a powder which contains 80 wt % solid [S,S] EDDS as an acid, and water of crystallisation.

Methylglycinediacetic acid (MGDA) has the structure shown in formula (C):

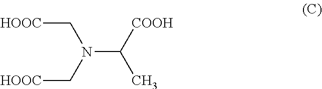

MGDA may have the structure shown in formula (C) and/or the same structure in which a number of the acidic protons have been replaced, i.e. in which 1, 2 or 3 the acid groups have been neutralised or partially neutralised. It may be present as a free acid or a salt or complex thereof.

When the chelating agent is MGDA it may be present as either enantiomer or a mixture thereof. Preferably it is present as a racemic mixture.

MGDA is commercially available as a solution comprising 40 wt % of the trisodium salt and is sold under the trade mark Trilon M.

Glutamic acid N,N-diacetic acid (GLDA) has the structure shown in formula (D):

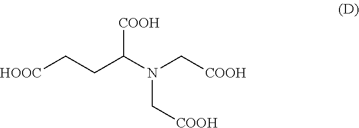 [SMILES: O=C(O)CCC(C(=O)O)N(CC(=O)O)CC(=O)O]

When the chelating agent comprises GLDA, it may have the structure shown in formula (D) and/or the same structure in which a number of the acidic protons have been replaced, i.e. in which 1, 2, 3 or 4 of the acid groups have been neutralised or partially neutralised. The GLDA may be present as a free acid or a salt or complex thereof.

GLDA may be present as either enantiomer or a mixture thereof. Preferably at least 50% is present as [S]-GLDA, preferably at least 70%, more preferably at least 90%, most preferably at least 95 wt %, for example about 98 wt %. Suitably, the GLDA consists essentially of the S enantiomer.

GLDA is commercially available as a solution comprising 38 wt % of the tetrasodium salt and is sold under the trade mark Dissolvine GL-38.

Diethylene triamine pentaacetic acid (DTPA) has the structure shown in formula (E):

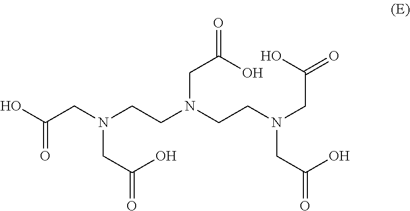 [SMILES: O=C(O)CN(CCN(CC(=O)O)CC(=O)O)CCN(CC(=O)O)CC(=O)O]

When the chelating agent comprises DTPA, it may have the structure shown in formula (E) or in a form having the same structure in which a number of the hydrogen atoms have been replaced. Thus DTPA may comprise salts in which 1, 2, 3, 4 or 5 of the acid groups have been neutralised or partially neutralised.

Suitable salts of DTPA include the salt of an alkali metal, an alkaline earth metal, ammonia or a suitable amine.

When a monovalent counterion is used the salt may be the monosalt, the disalt, the trisalt, the tetra salt or the pentasalt. For a divalent cation the monosalt or disalt may be present. Mixed salts may also exist, for example, the disodium magnesium salt or the sodium magnesium salt may be present. Preferably the counterion(s) to the DTPA residue is/are selected from one or more of sodium, magnesium, calcium, potassium, lithium, ammonium, and a quaternary ammonium ion.

Preferably when the chelating agent is DTPA it is present as the pentasodium salt.

Iminodisuccinic acid (IDS) has the structure shown in formula (F):

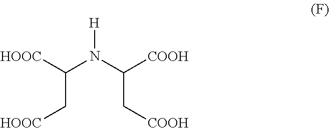 [SMILES: O=C(O)CC(NC(CC(=O)O)C(=O)O)C(=O)O]

When the chelating agent comprises IDS it may have the structure shown in formula (F) and/or the same structure in which a number of the acidic protons have been replaced, i.e. in which 1, 2, 3 or 4 of the acid groups have been neutralised or partially neutralised. The IDS may be present as a free acid or a salt or complex thereof.

IDS or a salt thereof may be present as either enantiomer or a mixture thereof. Preferably it is present as a racemic mixture.

IDS is commercially available as a solution comprising 34 wt % of the tetrasodium salt and is sold under the trade mark Baypure CX100.

L-aspartic acid diacetic acid (ASDA) is a structural isomer of IDS and has the structure shown in formula (G):

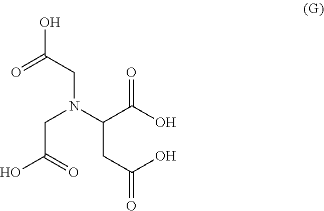 [SMILES: O=C(O)CC(C(=O)O)N(CC(=O)O)CC(=O)O]

When the chelating agent comprises ASDA it may have the structure shown in formula (G) and/or the same structure in which a number of the acidic protons have been replaced, i.e. in which 1, 2, 3 or 4 of the acid groups have been neutralised or partially neutralised. The ASDA may be present as a free acid or a salt or complex thereof.

Hydroxyethylethylenediaminetriacetic acid (known as HEEDTA or HEDTA) has the structure shown in formula (H):

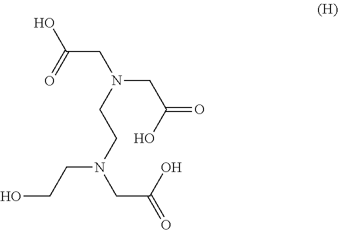 [SMILES: O=C(O)CN(CCO)CCN(CC(=O)O)CC(=O)O]

When the chelating agent comprises HEDTA it may have the structure shown in formula (H) and/or the same structure in which a number of the acidic protons have been replaced, i.e. in which 1, 2 or 3 of the acid groups have been neutralised or partially neutralised. The HEDTA may be present as a free acid or a salt or complex thereof.

HEDTA is commercially available from Akzo Nobel as the trisodium salt under the trade mark Dissolvine H40.

Preferably, in the first and second aspects, the chelating agent is GLDA or a salt thereof. For example, GLDA may be provided as the trisodium or tetrasodium salt (preferably tetrasodium) of GLDA. GLDA is believed to be bioavailable and biodegradable.

In the first and second aspects, the at least one chelating agent comprising at least one aminopolycarboxylic acid or a salt thereof may be added to the surfactant composition in any suitable amount, such as in an amount of from 0.1 to 10 wt %, such as from 0.2 to 8 wt %, preferably from 0.5 to 5 wt %, more preferably from 1 to 3 wt %, based on the total weight of the surfactant composition.

The chelating agent comprising L-glutamic acid-N,N-diacetic acid or a salt thereof may be added to the surfactant composition in any suitable amount such as from 0.1 to 10 wt %, such as from 0.2 to 8 wt %, preferably from 0.5 to 5 wt %, more preferably from 1 to 3 wt %, based on the total weight of the surfactant composition.

In the first and second aspects, the surfactant composition may comprise at least one surfactant independently selected from an anionic surfactant, a non-ionic surfactant, a cationic surfactant and an amphoteric surfactant, or mixtures of two or more such surfactants.

In the first and second aspects, the surfactant composition may comprise at least one surfactant independently selected from an anionic surfactant and an amphoteric surfactant, or mixtures thereof.

In the first and second aspects, the surfactant composition may comprise two or more surfactants independently selected from an anionic surfactant, a non-ionic surfactant, a cationic surfactant and an amphoteric surfactant, or mixtures of two or more such surfactants.

In the first and second aspects, the surfactant composition may comprise two or more surfactants independently selected from an anionic surfactant and an amphoteric surfactant, or mixtures thereof.

Suitably, in the first and second aspects, the surfactant composition may comprise at least one anionic surfactant and at least one amphoteric surfactant. The surfactant composition may comprise the anionic and amphoteric surfactant in a weight ratio in the range of 8:1 to 4.5:1.

In the first and second aspects, the surfactant composition may comprise any suitable anionic surfactant. Such surfactants will be known to the person skilled in the art. The surfactant composition may comprise an anionic surfactant in any suitable amount, such as in an amount of from 15 to 98 wt %, for example from 25 to 95 wt %, preferably from 15 to 40 wt % or more preferably from 25 to 40 wt %, based on the total weight of the surfactant composition.

Amounts of components quoted herein in the surfactant composition relate to the surfactant composition comprising the chelating agent comprising at least one aminopolycarboxylic acid or a salt thereof.

Suitable anionic surfactants for use herein include salts of: fatty acids; alkoxylated carboxylic acids; ester carboxylates; ethoxylated ester carboxylates; mono- or dialkyl sulfates; mono- or dialkyl ether sulfates; lauryl ether sulfates; alkyl sulfonates; alkyl aryl sulfonates; primary alkane disulfonates; alkene sulfonates; hydroxyalkane sulfonates; isethionates; alkyl isethionates; acyl isethionates; acyl alkyl isethionates; alkyl glyceryl ether sulfonates; alpha-olefin sulfonates; alkyl phosphates; sulfonates of alkylphenolpolyglycol ethers; alkyl sulfopolycarboxylic acid esters; alkyl sulfosuccinates; alkyl ether sulfosuccinates; taurates; acyl taurates; acyl alkyl taurates; products of condensation of fatty acids with oxy- and aminoalkanesulfonic acids; sulfated derivatives of fatty acids and polyglycols; alkyl and acyl sarcosinates; sulfoacetates; alkyl phosphates; alkyl phosphate esters; acyl lactates; alkanolamides of sulfated fatty acids, lipoaminoacids and acyl substituted amino acids, for example acyl glycinates and acyl glutamates. Particularly exemplary salts, where applicable, are sodium, potassium, ammonium, magnesium and triethanolamine salts.

Illustrative examples of preferred anionic surfactants include sodium lauryl sulfate, sodium lauryl ether sulfate, sodium lauroyl methyl taurate, sodium methyl cocoyl taurate, sodium methyl oleoyl taurate, sodium cocoyl isethionate, sodium lauroyl isethionate, sodium cocoyl methyl isethionate, sodium lauroyl methyl isethionate, sodium lauroyl glycinate, sodium cocoyl glycinate, sodium lauryl sarcosinate, and disodium oleamido monoisopropanolamine (MIPA) sulfosuccinate.

Especially preferred anionic surfactants for use herein are acyl isethionates, acyl alkyl isethionates, acyl taurates and acyl alkyl taurates, such as sodium lauroyl methyl taurate, sodium methyl cocoyl taurate, sodium methyl oleoyl taurate, sodium cocoyl isethionate, sodium lauroyl isethionate, sodium cocoyl methyl isethionate and sodium lauroyl methyl isethionate.

Where the surfactant composition comprises an anionic surfactant, the anionic surfactant may comprise at least one acyl alkyl isethionate surfactant, such as an acyl alkyl isethionate surfactant of the formula (I):

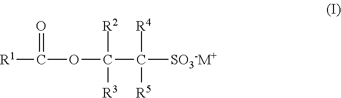

wherein R¹ represents an optionally substituted C₃-C₃₅ hydrocarbyl group; each of R², R³, R⁴ and R⁵ independently represents hydrogen or a C₁-C₄ alkyl group and wherein at least one of R², R³, R⁴ and R⁵ is not hydrogen; and M⁺ represents a cation.

Suitably, R¹ represents an optionally substituted C₃-C₃₅ alkyl, C₃-C₃₅ alkenyl, C₆-C₁₂ aryl, C₈-C₂₂ alkyl-C₆-C₁₂ aryl or C₆-C₁₂ aryl-C₈-C₂₂ alkyl group. More suitably, R¹ represents an optionally substituted C₃-C₃₅ alkyl or C₃-C₃₅ alkenyl group, especially an optionally substituted C₃-C₃₅ alkyl group. Most suitably, R¹ represents a C₃-C₃₅ alkyl or C₃-C₃₅ alkenyl group, especially a C₃-C₃₅ alkyl group.

Suitably, R¹ represents an optionally substituted C₃-C₃₅ alkyl or C₃-C₃₅ alkenyl group, such as an optionally substituted C₈-C₁₈ alkyl or C₈-C₁₈ alkenyl group.

Suitably, R¹ represents a C₃-C₃₅ alkyl or C₃-C₃₅ alkenyl group, such as a C₈-C₁₈ alkyl or C₈-C₁₈ alkenyl group.

Suitably, R¹ represents an optionally substituted C₅-C₃₀ alkyl group, such as an optionally substituted C₇-C₂₄ alkyl group, for example an optionally substituted C₇-C₂₁ alkyl group, preferably an optionally substituted C₇-C₁₇ alkyl group.

Suitably, R¹ represents a C₅-C₃₀ alkyl group, such as a C₇-C₂₄ alkyl group, for example a C₇-C₂₁ alkyl group, preferably a C₇-C₁₇ alkyl group.

R¹ is suitably the residue of a fatty acid. Fatty acids obtained from natural oils often include mixtures of fatty acids. For example, the fatty acid obtained from coconut oil contains a mixture of fatty acids including C₁₂ lauric acid, C₁₄ myristic acid, C₁₆ palmitic acid, C₈ caprylic acid, C₁₀ capric acid, C₁₈ stearic and C₁₈ oleic acid.

R¹ may include the residue of one or more naturally occurring fatty acids and/or of one or more synthetic fatty acids. For example, R¹ may consist essentially of the residue of a single fatty acid.

Examples of carboxylic acids from which R¹ may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behenic acid, erucic acid, docosahexanoic acid, lignoceric acid; naturally occurring fatty acids such as those obtained from coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Suitably R¹ comprises the residue of coco acid, the residue of mixed fatty acids derived from coconut oil and/or the residue of mixed fatty acids derived from palm kernel oil and/or the residue of mixed fatty acids derived from palm oil. More suitably, R¹ predominantly comprises the residue of a saturated fatty acid having 12 carbon atoms.

Preferably, R¹ is the residue of a hydrogenated fatty acid and therefore R¹ contains substantially no alkenyl groups. For example, R¹ may be provided by the residues resulting from the hydrogenation of coconut oil. Therefore, R¹ may be provided by the residues of a mixture of C₁₂ lauric acid, C₁₄ myristic acid, C₁₆ palmitic acid, C₈ caprylic acid, C₁₀ capric acid and C₁₈ stearic acid. R¹ may be a mixture of C₈, C₁₀, C₁₂, C₁₄, C₁₆ and C₁₈ alkyl groups, suitably straight chain alkyl groups, suitably derived from coconut oil. Fatty acids derived from coconut oil comprise at least 85 wt % fatty acids having 12 to 18 carbon atoms. Therefore, R¹ suitably comprises at least 85 wt % of C₁₂-C₁₈ alkyl groups. Suitably, in said mixtures, the majority of the R¹ groups are C₁₂ alkyl groups. R¹ may be provided by a topped fatty acid having a selected fatty acid chain length, wherein fatty acids with other chain lengths have been reduced or removed. For example, a topped fatty acid having predominantly C₁₂ alkyl groups as the R¹ groups may be used.

R¹ may be provided by the fatty acids obtained from coconut oil and/or palm kernel oil and/or palm oil.

The acyl alkyl isethionate surfactant of the formula (I) may be prepared by any of the methods disclosed in the prior art, for example see the methods described in WO94/09763 and WO2005/075623.

In some embodiments only a single acyl alkyl isethionate surfactant of the formula (I) may be present in the surfactant composition. In some embodiments a mixture of two or more acyl alkyl isethionate surfactants of the formula (I) may be present.

When any of R², R³, R⁴ and R⁵ represents an optionally substituted C₁-C₄ alkyl group, the alkyl group is suitably n-propyl, ethyl or methyl, such as ethyl or methyl, most preferably methyl.

Preferably one of the groups R², R³, R⁴ and R⁵ represents an optionally substituted C₁-C₄ alkyl group and the remaining groups represent hydrogen. For example, R² may represent an optionally substituted C₁-C₄ alkyl group and R³, R⁴ and R⁵ may all represent hydrogen. For example, R⁴ may represent an optionally substituted C₁-C₄ alkyl group and R², R³ and R⁵ may all represent hydrogen.

Preferably, R² represents a C₁-C₄ alkyl group and R³, R⁴ and R⁵ all represent hydrogen. Preferably, R⁴ represents a C₁-C₄ alkyl group and R², R³ and R⁵ all represent hydrogen. Most preferably, R² represents a methyl group and R³, R⁴ and R⁵ all represent hydrogen. Most preferably, R⁴ represents a methyl group and R², R³ and R⁵ all represent hydrogen.

For example, the acyl alkyl isethionate surfactant of the formula (I) may be selected from one or more of sodium lauroyl methyl isethionate, sodium cocoyl methyl isethionate and sodium oleoyl methyl isethionate. Sodium cocoyl methyl isethionate is preferred.

Suitably, M⁺ represents a metal cation or an optionally substituted ammonium cation, preferably a metal cation. By “optionally substituted ammonium cation”, we mean to refer to an ammonium cation wherein the nitrogen atom may be substituted with from 1 to 4 optionally substituted hydrocarbyl groups. Suitable ammonium cations include NH₄⁺ and the ammonium cation of triethanolamine. Suitable metal cations include alkali metal cations, for example sodium, lithium and potassium cations, and alkaline earth metal cations, for example calcium and magnesium cations. Suitably, M⁺ represents an alkali metal cation or an optionally substituted ammonium cation. Preferably, M⁺ represents a zinc, potassium or sodium cation. Most preferably, M⁺ represents a sodium cation.

The skilled person will appreciate that when M⁺ is a divalent metal cation two moles of anion will be present for each mole of cation.

The acyl alkyl isethionate surfactant of formula (I) may comprise the reaction product of sodium methyl isethionate and a fatty acid, that is a compound of formula R¹COOCHR²CHR⁴SO₃⁻M⁺ in which one of R² and R⁴ is methyl and the other is hydrogen. Mixtures of these isomers may be present.

The surfactant composition may include a mixture of more than one acyl alkyl isethionate surfactant of formula (I). For example, an isomeric mixture of acyl alkyl isethionate surfactants of formula (I) may be present. Such a mixture may include, for example an acyl alkyl isethionate surfactant in which R² represents a C₁-C₄ alkyl group (suitably methyl) and R³, R⁴ and R⁵ are all hydrogen and an acyl alkyl isethionate surfactant in which R⁴ represents a C₁-C₄ alkyl group (suitably methyl) and R², R³ and R⁵ are all hydrogen.

In particular, the surfactant composition may comprise a mixture of isomers, that is a compound of formula R¹COOCH₂CHR⁴SO₃⁻M⁺ in which R⁴ represents a C₁-C₄ alkyl group (preferably methyl) and a compound of formula R¹COOCHR²CH₂SO₃⁻M⁺ in which R² represents a C₁-C₄ alkyl group (preferably methyl).

Suitably such mixtures comprise approximately 90% of compounds in which R² is methyl and R⁴ is hydrogen and approximately 10% of compounds in which R² is hydrogen and R⁴ is methyl.

The surfactant composition may comprise at least one acyl alkyl isethionate in any suitable amount, such as in an amount of from 15 to 98 wt %, for example from 25 to 95 wt %, preferably from 15 to 40 wt % or more preferably from 25 to 40 wt %, based on the total weight of the surfactant composition.

In the first and second aspects, the surfactant composition may comprise any suitable amphoteric surfactant. Such surfactants will be known to the person skilled in the art. The surfactant composition may comprise an amphoteric surfactant in any suitable amount, such as in an amount of from 3 to 12 wt %, based on the total weight of the surfactant composition.

Suitable amphoteric surfactants for use herein include betaines (including sultaines or sulfobetaines) and amphoacetates. Preferred amphoteric surfactants include amido betaines (including amido sultaines or amido sulfobetaines) and amphoacetates.

Suitable amphoacetates include diamphoacetates. Amphoacetates generally conform to the following formula (II):

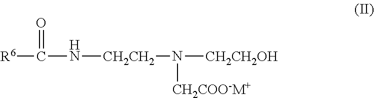

Diamphoacetates generally conform to the following formula (III):

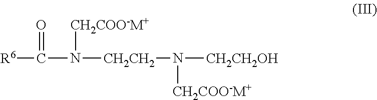

wherein R⁶ is an aliphatic group of 7 to 22 carbon atoms and M⁺ is a cation such as sodium, potassium, ammonium, or substituted ammonium.

Suitable acetate-based amphoteric surfactants include lauroamphoacetate; alkyl amphoacetate; cocoampho(di)acetate; cocoamphoacetate; cocoamphodiacetate; sodium cocoamphoacetate; disodium cocoamphodiacetate; disodium capryloamphodiacete; disodium lauroamphoacetate; sodium lauroamphoacetate and disodium wheatgerm amphodiacetate.

When the surfactant composition comprises an amphoteric surfactant, the amphoteric surfactant may comprise at least one betaine.

A suitable betaine may be a betaine (or amido betaine when n is 1) of the formula (IV):

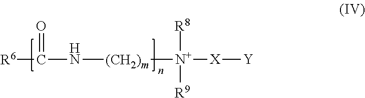

wherein R⁷ is a C₅ to C₃₀ alkyl or alkenyl group, R⁸ and R⁹ are each independently alkyl, hydroxyalkyl or carboxyalkyl of 1 to 6 carbon atoms; m is 2 to 4; n is 0 or 1; X is alkylene of 1 to 6 carbon atoms optionally substituted with hydroxyl; and Y is —CO₂— or —SO₃—.

Preferably R⁷ is a C₁₀ to C₂₀ alkyl or alkenyl group. R⁷ may be a mixture of alkyl groups. Suitably at least half, preferably at least three quarters, of the groups R⁷ by mole have 10 to 14 carbon atoms based on the total moles of betaine present.

R⁷ may be a mixture of alkyl groups derived from coconut or palm kernel oil.

R⁸ and R⁹ are preferably methyl.

A suitable betaine may be an alkyl betaine of the formula (V):

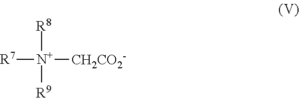

wherein R⁷, R⁸ and R⁹ are as defined previously.

The betaine may comprise an alkyl amido betaine of the formula (VI):

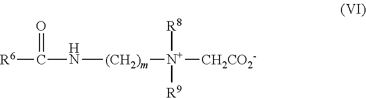

wherein R⁷, R⁸ and R⁹ are as defined previously; and m is 2 or 3.

The betaine may comprise a sultaine (or sulfobetaine) of the formulae (VII) or (VIII):

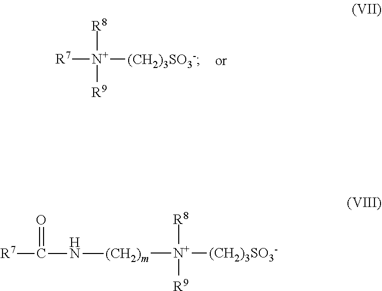

wherein R⁷, R⁸ and R⁹ are as defined previously; and m is 2 or 3;
or variants of these in which —(CH₂)₃SO₃— is replaced by

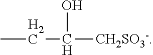

Suitable betaine surfactants include alkylamido betaine; alkyl betaine; C_{12/14} alkyldimethyl betaine; cocoamidopropylbetaine; tallow bis(hydroxyethyl) betaine; hexadecyldimethylbetaine; cocodimethylbetaine; alkyl amido propyl sulfo betaine; alkyl dimethyl amine betaine; coco amido propyl dimethyl betaine; alkyl amido propyl dimethyl amine betaine; cocamidopropyl betaine; lauryl betaine; laurylamidopropyl betaine; coco amido betaine; lauryl amido betaine; alkyl amino betaine; alkyl amido betaine; coco betaine; lauryl betaine; dimethicone propyl PG-betaine; oleyl betaine; N-alkyldimethyl betaine; coco biguanide derivative, C₈ amido betaine; C₁₂ amido betaine; lauryl dimethyl betaine; alkylamide propyl betaine; amido betaine; alkyl betaine; cetyl betaine; oleamidopropyl betaine; isostearamidopropyl betaine; lauramidopropyl betaine; 2-alkyl-N-carboxymethyl-N-hydroxyethyl imidazolinium betaine; 2-alkyl-N-carboxyethyl-N-hydroxyethyl imidazolinium betaine; 2-alkyl-N-carboxymethyl-N-hydroxyethyl imidazolinium betaine; N-alkyl amidopropyl-N,N-dimethyl-N-(3-sulfopropyl)-ammonium-betaine; N-alkyl-N,N-dimethyl-N-(3-sulfopropyl)-ammonium-betaine; cocodimethyl betaine; apricotamidopropyl betaine; isostearamidopropyl betaine; myristamidopropyl betaine; palmitamidopropyl betaine; cocamidopropyl hydroxy sultaine; undecylenamidopropyl betaine; cocoamidosulfobetaine; alkyl amido betaine; C_{12/18} alkyl amido propyl dimethyl amine betaine; lauryldimethyl betaine; ricinol amidobetaine; tallow amidobetaine.

Suitably, the betaine comprises an amido betaine, preferably a cocoamidopropyl betaine.

The at least one betaine may be present in the surfactant composition in any suitable amount, such as in an amount of from 3 to 12 wt % based on the total weight of the surfactant composition.

In the first and second aspects, the surfactant composition may comprise any suitable cationic surfactant. Such surfactants will be known to the person skilled in the art. The surfactant composition may comprise a cationic surfactant in any suitable amount, such as in an amount of from 15 to 40 wt % or from 20 to 30 wt %, based on the total weight of the surfactant composition.

Suitable cationic surfactants for use herein are typically based on fatty amine derivates or phosphonium quaternary ions, and quaternary ammonium compounds.

Suitable cationic surfactants for use herein include tertiary amine salts, mono alkyl trimethyl ammonium chloride, mono alkyl trimethyl ammonium methyl sulphate, dialkyl dimethyl ammonium chloride, dialkyl dimethyl ammonium methyl sulphate, trialkyl methyl ammonium chloride and trialkyl methyl ammonium methyl sulfate.

In the first and second aspects, the surfactant composition may comprise any suitable non-ionic surfactant. Such surfactants will be known to the person skilled in the art. The surfactant composition may comprise a non-ionic surfactant in any suitable amount, such as in an amount of from 15 to 40 wt % or from 20 to 30 wt %, based on the total weight of the surfactant composition.

Suitable non-ionic surfactants for use herein include alcohol ethoxylates and ethylene oxide/propylene oxide copolymer derived surfactants, sugar esters, especially sorbitan esters, alkyl polyglucosides, fatty acid ethoxylates or polyethylene glycol esters and partial esters, alkanolamides and amineoxides.

For example, suitable non-ionic surfactants for use herein include fatty acid alkanolamides, ethylene glycol stearate and ethylene glycol distearate.

Further suitable non-ionic surfactants include the reaction product of compounds having a hydrophobic group and a reactive hydrogen atom, for example aliphatic alcohols, acids, amides or alkyl phenols with alkylene oxides, especially ethylene oxide either alone or with propylene oxide (for example alkyl (C₆-C₂₂) phenol-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine); long chain tertiary amine oxides, long chain tertiary phosphine oxides and dialkyl sulfoxides; alkyl amine oxides, alkyl amido amine oxides; alkyl tertiary phosphine oxides; alkoxyl alkyl amines; sorbitan; sorbitan esters; sorbitan ester alkoxylates; glycerol ester alkoxylates; sucrose esters; sugar amides, such as a polysaccharide amide; lactobionamides; and alkyl polysaccharide nonionic surfactants, for example alkylpolyglycosides. Preferred non-ionic surfactants are sucroglycerides and ethyoxylated fatty alcohols, especially those derived from lauryl, cetylstearyl, stearyl, cetyl and oleocetyl alcohols.

In the first and second aspects, the surfactant composition (to which the chelating agent is added) may comprise:
(i) at least one acyl alkyl isethionate surfactant of the formula (I):

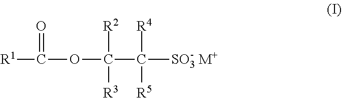

wherein R¹ represents an optionally substituted C₃-C₃₅ hydrocarbyl group; each of R², R³, R⁴ and R⁵ independently represents hydrogen or a C₁-C₄ alkyl group and wherein at least one of R², R³, R⁴ and R⁵ is not hydrogen; and M⁺ represents a cation; and
(ii) at least one amphoteric surfactant.

In the first and second aspects, the surfactant composition (to which the chelating agent is added) may comprise:
(i) at least one acyl alkyl isethionate surfactant of the formula (I):

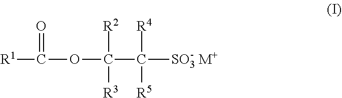

wherein R¹ represents an optionally substituted C₃-C₃₅ hydrocarbyl group; each of R², R³, R⁴ and R⁵ independently represents hydrogen or a C₁-C₄ alkyl group and wherein at least one of R², R³, R⁴ and R⁵ is not hydrogen; and M⁺ represents a cation; and
(ii) at least one betaine surfactant.

In the first and second aspects, the surfactant composition (to which the chelating agent is added) may comprise:
(i) at least one acyl alkyl isethionate surfactant of the formula (I):

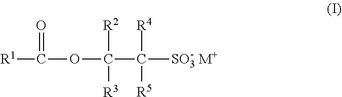

wherein R¹ represents an optionally substituted C₃-C₃₅ hydrocarbyl group; each of R², R³, R⁴ and R⁵ independently represents hydrogen or a C₁-C₄ alkyl group and wherein at least one of R², R³, R⁴ and R⁵ is not hydrogen; and M⁺ represents a cation; and
(ii) at least one amido betaine, preferably at least one cocoamidopropyl betaine

Preferably, in the first and second aspects, the surfactant composition (to which the chelating agent is added) may comprise:
(i) at least one acyl alkyl isethionate selected from sodium lauroyl methyl isethionate and sodium cocoyl methyl isethionate; and
(ii) cocamidopropyl betaine.

The surfactant composition may be in a liquid form. Suitably, the surfactant composition may be in a liquid form at room temperature and pressure (i.e. 25° C. and 1 atm).

The surfactant composition may further comprise at least one solvent. Any suitable solvent may be used, such as an aqueous solvent, for example water.

The at least one solvent may be present in the surfactant composition in any suitable amount, such as in an amount of from 40 to 95 wt %. such as from 40 to 75 wt %, preferably from 45 to 70 wt % or more preferably from 45 to 60 wt %, based on the total weight of the surfactant composition.

The surfactant composition may further comprise any suitable additional ingredients. Examples of suitable such additional ingredients include pH adjusters (such as citric acid), fragrances, dyes and preservatives (such as sodium benzoate), as would be known to persons skilled in the art. The surfactant composition may comprise a fatty acid as an additional ingredient, such as a fatty acid that is present as an impurity in a surfactant comprised in the surfactant composition. Suitably, when the surfactant composition comprises a fatty acid, the fatty acid is present in an amount of less than 5 wt %, for example less than 3 wt %, based on the total weight of the surfactant composition.

The use and method of the first and second aspects may act to provide a transparent surfactant composition comprising at least one acyl alkyl isethionate in any suitable amount, at least one amphoteric surfactant in any suitable amount, and at least one chelating agent, wherein the chelating agent comprises at least one aminopolycarboxylic acid or a salt thereof. Suitably, the use and method of the first and second aspects may act to provide a transparent surfactant composition comprising at least one acyl alkyl isethionate in an amount of from 15 to 98 wt % (for example 15 to 95 wt %), an amphoteric surfactant in an amount of from 3 to 12 wt %, and at least one chelating agent in an amount of from 0.1 to 10 wt % based on the total weight of the transparent surfactant composition, wherein the chelating agent comprises at least one aminopolycarboxylic acid or a salt thereof.

The use and method of the first and second aspects may act to provide a transparent surfactant composition of the third aspect of the present invention as described herein. For example, the use and method of the first and second aspects may act to provide a transparent surfactant composition comprising:
(i) from 15 to 98 wt % (for example 15 to 95 wt %) of at least one acyl alkyl isethionate surfactant of the formula (I):

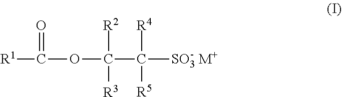

wherein R¹ represents an optionally substituted C₃-C₃₅ hydrocarbyl group;
each of R², R³, R⁴ and R⁵ independently represents hydrogen or a C₁-C₄ alkyl group and
wherein at least one of R², R³, R⁴ and R⁵ is not hydrogen; and M⁺ represents a cation;
(ii) from 3 to 12 wt % of at least one amphoteric surfactant; and
(iii) from 0.1 to 10 wt % of at least one chelating agent, wherein the chelating agent comprises at least one aminopolycarboxylic acid or a salt thereof.

In the first and second aspects, the surfactant composition and/or the resulting transparent surfactant composition may be a concentrate composition. Such a concentrate composition may be diluted with other components to produce a formulated composition. Concentrate compositions are useful as they take up less space in storage and require less energy to transport than formulated compositions. It is especially beneficial for a concentrate composition to be transparent as it enables the composition to more easily be formulated into transparent compositions. It is also especially beneficial for a concentrate composition to not discolour over time as concentrate compositions may be stored for long periods prior to formulation and discoloured concentrates will affect the colour of formulated compositions.

According to a third aspect of the present invention, there is provided a transparent surfactant composition comprising:
(i) from 15 to 98 wt % of at least one acyl alkyl isethionate surfactant of the formula (I):

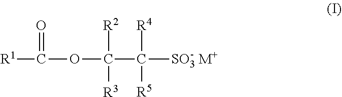

wherein R¹ represents an optionally substituted C₃-C₃₅ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
(ii) from 3 to 12 wt % of at least one amphoteric surfactant; and
(iii) from 0.1 to 10 wt % of at least one chelating agent, wherein the chelating agent comprises at least one aminopolycarboxylic acid or a salt thereof.

The third aspect may provide a transparent surfactant composition comprising:
(i) from 15 to 95 wt % of at least one acyl alkyl isethionate surfactant of the formula (I):

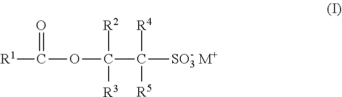

wherein R¹ represents an optionally substituted C₃-C₃₅ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
(ii) from 3 to 12 wt % of at least one amphoteric surfactant; and
(iii) from 0.1 to 10 wt % of at least one chelating agent, wherein the chelating agent comprises at least one aminopolycarboxylic acid or a salt thereof.

Features of the at least one acyl alkyl isethionate surfactant of the formula (I), of the at least one amphoteric surfactant (including betaines) and of the chelating agent in relation to the third aspect of the invention are as disclosed herein in relation to the first and second aspects of the invention.

Suitably, in the third aspect, the transparent surfactant composition may comprise the acyl alkyl isethionate surfactant of the formula (I) and the amphoteric surfactant in a weight ratio in the range of 8:1 to 4.5:1.

The transparent surfactant composition of the third aspect may be produced by the method or use of the first or second aspect of the present invention.

The total amount of surfactant in the transparent surfactant composition of the third aspect (including the acyl alkyl isethionate surfactant of the formula (I) and the amphoteric surfactant) may be in the range of from 18 to 99.9 wt %, such as 20 to 95 wt % or preferably from 25 to 50 wt % or more preferably from 30 to 45 wt %, based on the total weight of the surfactant composition.

Suitably, in the third aspect, at least 50 wt % of the total surfactants present are acyl alkyl isethionate surfactants of formula (I). Suitably, from 50 to 90 wt %, such as from 65 to 90 wt %, preferably from 75 to 90 wt % of the total surfactants present are acyl alkyl isethionate surfactants.

The transparent surfactant composition of the third aspect may be a concentrate surfactant composition. Suitably, when the transparent surfactant composition is a concentrate surfactant composition, the total amount of surfactant may be at least 25 wt %, such as at least 30 wt % based on the total weight of the concentrate surfactant composition. For example, the total amount of surfactant may be in the range of from 25 to 60 wt %, suitably from 30 to 50 wt %, preferably from 30 to 45 wt %, based on the total weight of the concentrate surfactant composition.

In the transparent surfactant composition of the third aspect, the surfactants present may consist essentially of (or consist of) at least one acyl alkyl isethionate surfactant of the formula (I) and at least one amphoteric surfactant (such as a least one betaine surfactant).

The third aspect may provide a transparent surfactant composition comprising:
(i) from 15 to 98 wt % of at least one acyl alkyl isethionate surfactant of the formula (I):

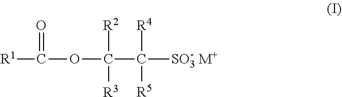

wherein R¹ represents an optionally substituted C₃-C₃₅ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
(ii) from 3 to 12 wt % of at least one betaine surfactant, such as at least one amido betaine, preferably at least one cocoamidopropyl betaine; and
(iii) from 0.1 to 10 wt % of at least one chelating agent wherein the chelating agent comprises at least one aminopolycarboxylic acid or a salt thereof.

The third aspect may provide a transparent surfactant composition consisting essentially of or consisting of:
(i) from 15 to 98 wt % of at least one acyl alkyl isethionate surfactant of the formula (I):

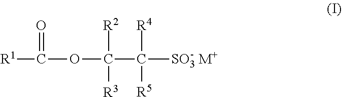

wherein R¹ represents an optionally substituted C₃-C₃₅ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
(ii) from 3 to 12 wt % of at least one betaine surfactant, such as at least one amido betaine, preferably at least one cocoamidopropyl betaine; and
(iii) from 0.1 to 10 wt % of at least one chelating agent wherein the chelating agent comprises at least one aminopolycarboxylic acid or a salt thereof.

The third aspect may provide a transparent surfactant composition comprising:
(i) from 15 to 95 wt % of at least one acyl alkyl isethionate surfactant of the formula (I):

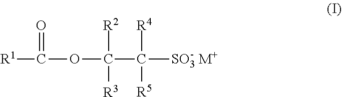

wherein R¹ represents an optionally substituted C₃-C₃₅ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
(ii) from 3 to 12 wt % of at least one betaine surfactant, such as at least one amido betaine, preferably at least one cocoamidopropyl betaine; and
(iii) from 0.1 to 10 wt % of at least one chelating agent wherein the chelating agent comprises at least one aminopolycarboxylic acid or a salt thereof.

The third aspect may provide a transparent surfactant composition consisting essentially of or consisting of:
(i) from 15 to 95 wt % of at least one acyl alkyl isethionate surfactant of the formula (I):

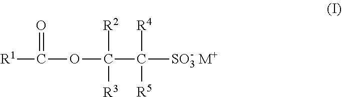

wherein R¹ represents an optionally substituted C₃-C₃₅ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
(ii) from 3 to 12 wt % of at least one betaine surfactant, such as at least one amido betaine, preferably at least one cocoamidopropyl betaine; and
(iii) from 0.1 to 10 wt % of at least one chelating agent wherein the chelating agent comprises at least one aminopolycarboxylic acid or a salt thereof.

In the third aspect, the at least one aminopolycarboxylic acid may be selected from one or more of ethylenediamine-N,N′-diglutaric acid (EDDG), ethylenediamine-N,N′-dimalonic acid (EDDM), iminodisuccinic acid (IDS), ethylenediaminetetraacetic acid (EDTA), ethylenediaminedisuccinic acid (EDDS), methylglycinediacetic acid (MGDA), L-glutamic acid-N,N-diacetic acid (GLDA), diethylene triamine pentaacetic acid (DTPA), L-aspartic acid diacetic acid (ASDA), hydroxyethyl ethylenediaminetriacetic acid (HEDTA), iminodifumaric acid (IDF), iminoditartaric acid (IDT), iminodimaleic acid (IDMAL), iminodimalic acid (IDM), ethylenediaminedifumaric acid (EDDF), ethylenediaminedimalic acid (EDDM), ethylenediamineditartaric acid (EDDT) and ethylenediaminedimaleic acid (EDDMAL), or a salt thereof.

In the third aspect, the at least one aminopolycarboxylic acid may be selected from one or more of ethylenediamine-N,N′-diglutaric acid (EDDG), ethylenediamine-N,N′-dimalonic acid (EDDM), iminodisuccinic acid (IDS), ethylenediaminetetraacetic acid (EDTA), ethylenediaminedisuccinic acid (EDDS), methylglycinediacetic acid (MGDA) and L-glutamic acid-N,N-diacetic acid (GLDA), preferably from EDTA, EDDS, MGDA and GLDA, or a salt thereof.

Suitable salts of the aminopolycarboxylic acid include amine salts, alkaline earth metal salts or alkali metal salts. Suitable amine salts include those of ammonia and triethanolamine. Suitable alkaline earth metal salts include those of magnesium and calcium. Suitable alkali metal salts include sodium, lithium and potassium salts. Preferably the chelating agent is a provided as a sodium salt. Suitable salts include partial salts where one or more but not all of the acid groups are present as salts.

Preferably, in the third aspect, the chelating agent is GLDA, for example the tetrasodium salt of GLDA.

In the third aspect, the transparent surfactant composition may comprise the at least one acyl alkyl isethionate surfactant of the formula (I) in an amount of from 15 to 95 wt %, preferably from 15 to 40 wt %, based on the total weight of the surfactant composition.

In the third aspect, the transparent surfactant composition may comprise the at least one acyl alkyl isethionate surfactant of the formula (I) in an amount of from 25 to 95 wt %, preferably from 15 to 40 wt % or more preferably from 25 to 40 wt %, based on the total weight of the surfactant composition.

In the third aspect, the transparent surfactant composition may comprise the at least one chelating agent comprising at least one aminopolycarboxylic acid or a salt thereof in an amount of from 0.2 to 8 wt %, preferably from 0.5 to 5 wt %, preferably from 1 to 3 wt %, based on the total weight of the surfactant composition.

Preferably, the transparent surfactant composition of the third aspect may comprise:
(i) from 25 to 95 wt % of at least one acyl alkyl isethionate selected from sodium lauroyl methyl isethionate and sodium cocoyl methyl isethionate;
(ii) from 3 to 12 wt % of an amido betaine; and
(iii) from 0.1 to 10 wt % of at least one aminopolycarboxylic acid or a salt thereof.

More preferably, the transparent surfactant composition of the third aspect may comprise:
(i) from 25 to 95 wt % of at least one acyl alkyl isethionate selected from sodium lauroyl methyl isethionate and sodium cocoyl methyl isethionate;
(ii) from 3 to 12 wt % of an amido betaine; and
(iii) from 0.1 to 10 wt % of L-glutamic acid-N,N-diacetic acid or a salt thereof.

Preferably, the transparent surfactant composition of the third aspect may comprise:
(i) from 25 to 95 wt % of at least one acyl alkyl isethionate selected from sodium lauroyl methyl isethionate and sodium cocoyl methyl isethionate;
(ii) from 3 to 12 wt % of an amido betaine; and
(iii) from 0.5 to 5 wt % of at least one aminopolycarboxylic acid or a salt thereof.

More preferably, the transparent surfactant composition of the third aspect may comprise:
(i) from 25 to 95 wt % of at least one acyl alkyl isethionate selected from sodium lauroyl methyl isethionate and sodium cocoyl methyl isethionate;
(ii) from 3 to 12 wt % of an amido betaine; and
(iii) from 0.5 to 5 wt % of L-glutamic acid-N,N-diacetic acid or a salt thereof.

Preferably, the transparent surfactant composition of the third aspect may comprise:
(i) from 25 to 95 wt % of at least one acyl alkyl isethionate selected from sodium lauroyl methyl isethionate and sodium cocoyl methyl isethionate;
(ii) from 3 to 12 wt % of cocamidopropyl betaine; and
(iii) from 0.1 to 10 wt % of at least one aminopolycarboxylic acid or a salt thereof.

More preferably, the transparent surfactant composition of the third aspect may comprise:
(i) from 25 to 95 wt % of at least one acyl alkyl isethionate selected from sodium lauroyl methyl isethionate and sodium cocoyl methyl isethionate;
(ii) from 3 to 12 wt % of cocamidopropyl betaine; and
(iii) from 0.1 to 10 wt % of L-glutamic acid-N,N-diacetic acid or a salt thereof.

Preferably, the transparent surfactant composition of the third aspect may comprise:
(i) from 25 to 95 wt % of at least one acyl alkyl isethionate selected from sodium lauroyl methyl isethionate and sodium cocoyl methyl isethionate;
(ii) from 3 to 12 wt % of cocamidopropyl betaine; and
(iii) from 0.5 to 5 wt % of at least one aminopolycarboxylic acid or a salt thereof.

More preferably, the transparent surfactant composition of the third aspect may comprise:
(i) from 25 to 95 wt % of at least one acyl alkyl isethionate selected from sodium lauroyl methyl isethionate and sodium cocoyl methyl isethionate;
(ii) from 3 to 12 wt % of cocamidopropyl betaine; and
(iii) from 0.5 to 5 wt % of L-glutamic acid-N,N-diacetic acid or a salt thereof.

Preferably, the transparent surfactant composition of the third aspect may comprise:
(i) from 15 to 40 wt % of at least one acyl alkyl isethionate selected from sodium lauroyl methyl isethionate and sodium cocoyl methyl isethionate;
(ii) from 3 to 12 wt % of cocamidopropyl betaine; and
(iii) from 1 to 3 wt % of at least one aminopolycarboxylic acid or a salt thereof.

More preferably, the transparent surfactant composition of the third aspect may comprise:
(i) from 15 to 40 wt % of at least one acyl alkyl isethionate selected from sodium lauroyl methyl isethionate and sodium cocoyl methyl isethionate;
(ii) from 3 to 12 wt % of cocamidopropyl betaine; and
(iii) from 1 to 3 wt % of L-glutamic acid-N,N-diacetic acid or a salt thereof.

The transparent surfactant composition of the third aspect may remain transparent without substantial discolouration upon storage for a period of time, such as a period of 1 to 2 years, or a period of 1 to 3 years, at ambient temperature or such as a period of at least 2 weeks, for example at least 4, 6 or 8 weeks, or at least 4, 6, 8 or 12 weeks.

The transparent surfactant composition of the third aspect may remain transparent without substantial discolouration upon storage for a period of time, such as a period of time of at least 4, 6 or 8 weeks, or at least 4, 6, 8 or 12 weeks, at a temperature above ambient temperature, such as a temperature of 40 to 50° C., for example about 45° C.

The transparent surfactant composition of the third aspect may remain transparent without substantial discolouration upon storage for a period of time, at least 4, 6 or 8 weeks, or at least 4, 6, 8 or 12 weeks, at a temperature below ambient temperature, such as a temperature of 0 to 10° C., for example about 5° C.

The transparent surfactant composition of the third aspect may remain transparent without substantial discolouration upon storage for a period of at least 2 weeks (for example at least 4, 6 or 8 weeks, or at least 4, 6, 8 or 12 weeks), for example at a temperature above ambient temperature and/or upon exposure to light.

The transparent surfactant composition of the third aspect may remain transparent without substantial discolouration upon storage for a period of at least 2 weeks (for example at least 4, 6 or 8 weeks, or at least 4, 6, 8 or 12 weeks), for example at a temperature below ambient temperature and/or upon exposure to light.

The transparent surfactant composition of the third aspect may have a transmittance value of 90% or greater and may have a yellowness index of 125 Hazen units or less (such as 80 Hazen units or less) upon storage for a period of at least 2 weeks (for example at least 4, 6 or 8 weeks, or at least 4, 6, 8 or 12 weeks), for example at a temperature above ambient temperature and/or upon exposure to light.

The transparent surfactant composition of the third aspect may have a transmittance value of 90% or greater and may have a yellowness index of 125 Hazen units or less (such as 80 Hazen units or less) upon storage for a period of at least 2 weeks (for example at least 4, 6 or 8 weeks, or at least 4, 6, 8 or 12 weeks), for example at a temperature below ambient temperature and/or upon exposure to light.

The transparent surfactant composition of the third aspect may remain transparent without substantial discolouration upon storage for a period of at least 2 weeks (for example at least 4, 6 or 8 weeks, or at least 4, 6, 8 or 12 weeks) at a temperature above ambient temperature and upon exposure to light.

The transparent surfactant composition of the third aspect may have a transmittance value of 90% or greater and may have a yellowness index of 125 Hazen units or less (such as 80 Hazen units or less) upon storage for a period of at least 2 weeks (for example at least 4, 6 or 8 weeks, or at least 4, 6, 8 or 12 weeks) at a temperature above ambient temperature and upon exposure to light.

The transparent surfactant composition of the third aspect may remain transparent without substantial discolouration upon storage for a period of at least 2 weeks (for example at least 4, 6 or 8 weeks, or at least 4, 6, 8 or 12 weeks) at a temperature above ambient temperature.

The transparent surfactant composition of the third aspect may have a transmittance value of 90% or greater and may have a yellowness index of 125 Hazen units or less (such as 80 Hazen units or less) upon storage for a period of at least 2 weeks (for example at least 4, 6 or 8 weeks, or at least 4, 6, 8 or 12 weeks) at a temperature above ambient temperature.

The transparent surfactant composition of the third aspect may remain transparent without substantial discolouration upon storage for a period of at least 2 weeks (for example at least 4, 6 or 8 weeks, or at least 4, 6, 8 or 12 weeks) at a temperature below ambient temperature and upon exposure to light.

The transparent surfactant composition of the third aspect may have a transmittance value of 90% or greater and may have a yellowness index of 125 Hazen units or less (such as 80 Hazen units or less) upon storage for a period of at least 2 weeks (for example at least 4, 6 or 8 weeks, or at least 4, 6, 8 or 12 weeks) at a temperature below ambient temperature and upon exposure to light.

The transparent surfactant composition of the third aspect may remain transparent without substantial discolouration upon storage for a period of at least 2 weeks (for example at least 4, 6 or 8 weeks, or at least 4, 6, 8 or 12 weeks) at a temperature below ambient temperature.

The transparent surfactant composition of the third aspect may have a transmittance value of 90% or greater and may have a yellowness index of 125 Hazen units or less (such as 80 Hazen units or less) upon storage for a period of at least 2 weeks (for example at least 4, 6 or 8 weeks, or at least 4, 6, 8 or 12 weeks) at a temperature below ambient temperature.

The transparent surfactant composition of the third aspect may remain transparent without substantial discolouration upon storage for a period of 1 to 2 years, or a period of 1 to 3 years, at ambient temperature.

The transparent surfactant composition of the third aspect may have a transmittance value of 90% or greater and a yellowness index of 125 Hazen units or less (such as 80 Hazen units or less) upon storage for a period of 1 to 2 years, or a period of 1 to 3 years, at ambient temperature.

The transparent surfactant composition of the third aspect may remain transparent without substantial discolouration upon storage for a period of at least 2 weeks (for example at least 4, 6 or 8 weeks, or at least 4, 6, 8 or 12 weeks) upon exposure to light.

The transparent surfactant composition of the third aspect may have a transmittance value of 90% or greater and a yellowness index of 125 Hazen units or less (such as 80 Hazen units or less) upon storage for a period of at least 2 weeks (for example at least 4, 6 or 8 weeks, or at least 4, 6, 8 or 12 weeks) upon exposure to light.

The transparent surfactant composition of the third aspect may remain transparent without substantial discolouration upon storage in a sealed container at ambient temperature and without exposure to light for a period of at least 8 weeks, or at least 12 weeks, such as at least 1 year, preferably at least 2 years, such as at least 3 years.

The transparent surfactant composition of the third aspect may have a transmittance value of 90% or greater and may have a yellowness index of 125 Hazen units or less (such as 80 Hazen units or less) upon storage for a period of at least 1 year, preferably at least 2 years, such as at least 3 years, in a sealed container at ambient temperature and without exposure to light.

The transparent surfactant composition of the third aspect may remain transparent (for example having a turbidity value of less than 10 NTU) without substantial discolouration upon storage in a sealed container at 5° C. and without exposure to light for a period of at least 8 weeks, suitably for period of at least 12 weeks.

The transparent surfactant composition of the third aspect may remain transparent (for example having a turbidity value of less than 10 NTU) without substantial discolouration upon storage in a sealed container at 25° C. and without exposure to light for a period of at least 8 weeks, suitably for period of at least 12 weeks.

The transparent surfactant composition of the third aspect may remain transparent (for example having a turbidity value of less than 10 NTU) without substantial discolouration upon storage in a sealed container at 45° C. and without exposure to light for a period of at least 8 weeks, suitably for period of at least 12 weeks.

The transparent surfactant composition of the third aspect may be in a liquid form. Suitably, the transparent surfactant composition may be in a liquid form at room temperature and pressure (i.e. 25° C. and 1 atm).

The transparent surfactant composition of the third aspect may further comprise at least one solvent. Any suitable solvent may be used, such as an aqueous solvent, for example water.

The at least one solvent may be present in the transparent surfactant composition of the third aspect in any suitable amount, such as in an amount of from 40 to 95 wt %. such as from 40 to 75 wt %, preferably from 45 to 70 wt % or more preferably from 45 to 60 wt %, based on the total weight of the surfactant composition.

The transparent surfactant composition of the third aspect may further comprise at least one cationic surfactant, such as those based on fatty amine derivates or phosphonium quaternary ions, and quaternary ammonium compounds.

The transparent surfactant composition of the third aspect may further comprise any suitable additional ingredients. Examples of suitable such additional ingredients include pH adjusters (such as citric acid), fragrances, dyes and preservatives (such as sodium benzoate), as would be known to persons skilled in the art. The surfactant composition may comprise a fatty acid as an additional ingredient, such as a fatty acid that is present as an impurity in a surfactant comprised in the surfactant composition.

The surfactant compositions herein may have a pH of from 6 to 9, for example from 6 to 8.

According to a fourth aspect of the present invention, there is provided a personal or household cleaning composition comprising a transparent surfactant composition according to the third aspect.

The transparent surfactant composition according to the third aspect may be included in any suitable personal or household cleaning composition, as would be appreciated by persons skilled in the art. Examples of suitable personal or household cleaning compositions include bodywash, shampoo and face cleanser. Examples of suitable household cleaning compositions include hard surface cleaning products.

The personal and household cleaning compositions may include additional suitable components would be well known to persons skilled in the art and selected according to the particular purpose of the composition. Examples of suitable additional components include conditioning agents (including quaternary ammonium compounds, cationic polymers, silicones, synthetic or natural oils or resins etc), fatty alcohols, electrolytes or other rheology modifiers, opacifying/pearlising agents, scalp benefit agents, fragrances, dyes, UV filters, penetration enhancers (eg, propylene carbonate, benzyl alcohol etc), preservatives, antioxidants, emulsifiers, pH adjusting agents (for example lactic acid, sodium hydroxide, sodium phosphate and salts and buffers thereof), and buffers and styling polymers (eg polyvinylpyrrolidone etc).

The relative ratios of the components and the formulation of such compositions would be within the competence of the skilled person. For example, the personal or household cleaning composition of the fourth aspect may comprise from 1 to 30 wt % of the transparent surfactant composition according to the third aspect, based on the total weight of the personal or household cleaning composition.

According to a fifth aspect of the present invention, there is provided a method of preparing a transparent surfactant composition of the third aspect, the method comprising admixing the at least one acyl alkyl isethionate of the formula (I), the at least one betaine surfactant and the at least one chelating agent in the amounts set out in the third aspect.

The method of the fifth aspect may have any of the features and advantages described in relation to the first, second or third aspects.

### BRIEF DESCRIPTION OF THE FIGURES

For a better understanding of the invention, and to show how example embodiments may be carried into effect, reference will now be made to the accompanying FIGURES in which:

FIG. 1 shows the results of Example 1.

### EXAMPLES

For a better understanding of the invention, and to show how exemplary embodiments of the same may be brought into effect, reference will be made to the following non-limiting examples.

### Yellowness Index Measurement

### Method 1

The yellowness index was measured using a Lovibond® Comparator 2000+ instrument. This instrument determines the yellowness in Hazen or APHA units of a clear liquid sample, by visual comparison of a reference, the sample, and a standardized colour glass disc (a Nessleriser disc according to ASTM D1209). The method involves the following steps:
1. Fill one Nessler cylinder to just above the 50 mL mark with the sample under examination.
2. Stand the cylinder with the sample in the right-hand compartment of the Lovibond 2000+ comparator.
3. Fill the second Nessler cylinder in the same way with deionised water and place it in the left-hand compartment.
4. Insert the appropriate Nessleriser disc into the Lovibond 2000+ Comparator lid and turn on the light unit.
5. Rotate the disc until the closest colour match is obtained.
6. Record the value displayed as Hazen units. The higher the Hazen unit, the more yellow is the solution.

### Method 2

The yellowness index was measured using a HunterLab Vista Spectrophotometer instrument according to ASTM D5386. This instrument determines the yellowness in Hazen or APHA units of a liquid sample.

### Transparency Measurement

Transparency can be measured in different ways, such as by measuring the amount of light that passes through a sample, so as to provide a transmittance value (% T), or by measuring the scattering of light by a sample, so as to provide an indication of turbidity.

### Method 3—Transmittance Test Measurement

The transmittance was measured using a Photometer 7100 of Palintest® in the transmittance mode. Distilled water was used as blank. The transmittance value is recorded as % T or the transmittance of visible light through the sample. An opaque system will give a transmittance value of 0%. As used herein, a surfactant composition having a transmittance value of 90% or greater is considered to be transparent.

### Method 4—Turbidity Test Measurement

The turbidity was measured using a HunterLab Vista instrument that is standardized daily with DI water using NTU-10 mm index from 0-150 NTU in parallel with the APHA measurement. Three grams of surfactant sample blend was dispensed into a cuvette and measured for the initial turbidity value (D65/10). If any air bubbles were observed in the light path the sample was sonicated in a Branson 5800 instrument until all air was removed before the measurement.

Then the samples were placed into stability chambers (at 5, 25 and 45° C.) for the duration of the test and were removed for 4 hours on the day of the intermediate measurement. The samples were equilibrated in a 25° C. water bath for the duration of the 4 hours before taking the turbidity value (D65/10) measurement. Once the turbidity value (D65/10) was measured the samples were placed back into the temperature control chamber. The wavelength of light used by the HunterLab instrument for colour measurement is from Illuminant D65, which is used to mimic an average daylight distribution (including ultraviolet wavelength region) with a correlated colour temperature of about 6500 K.

As used herein, a surfactant composition having a turbidity value of less than 10 NTU is considered to be transparent.

### Example 1

The following liquid compositions were prepared by mixing the components listed in Table 1 at a temperature between 4° and 55° C. All amounts are given as a percentage by weight (wt %) of the active ingredient. The Blank is a surfactant composition without a chelating agent, Compositions 1 to 4 are transparent surfactant compositions according to the invention and Compositions 5 and 6 are comparative compositions.

TABLE 1

| | | Amount of active ingredient (wt %) | | | | | | |
|---|---|---|---|---|---|---|---|---|
| Composition | | Blank | 1 | 2 | 3 | 4 | 5 | 6 |
| Anionic | Sodium | 28.80 | 28.80 | 28.80 | 28.80 | 28.80 | 28.80 | 28.80 |
| surfactant | cocoyl | | | | | | | |
| | methyl | | | | | | | |
| | isethionate | | | | | | | |
| | (SCMI) ^{[1]} | | | | | | | |
| Amphoteric | Cocamido- | 6.17 | 6.17 | 6.17 | 6.17 | 6.17 | 6.17 | 6.17 |
| surfactant | propyl | | | | | | | |
| | betaine ^{[2]} | | | | | | | |
| Preservative | Sodium | 0.50 | 0.50 | 0.50 | 0.50 | 0.50 | 0.50 | 0.50 |
| | benzoate ^{[3]} | | | | | | | |
| pH adjuster | Citric acid | 0.15 | 0.15 | 0.15 | 0.15 | 0.15 | 0.15 | 0.15 |
| | solution ^{[4]} | | | | | | | |
| DI water | — | 57.5 | 56.3 | 56.3 | 56.3 | 56.3 | 56.3 | 56.3 |
| Chelating | GLDA | | 1.2 | | | | | |
| agent* | EDDS | | | 1.1 | | | | |
| | EDTA | | | | 1.1 | | | |
| | MGDA | | | | | 1.1 | | |
| | ATMP | | | | | | 1.1 | |
| | EDTMP | | | | | | | 1.1 |

| Transmittance (%) | 0 | 100 | 0 |
|---|---|---|---|
| Yellowness index | N/A | 60-70 | N/A |
| (Hazen units) | | | |

Components used as source of active ingredients:
^{[1]} SCMI was a commercially available source provided as solid flakes containing 80 wt % of active surfactant compound and containing 7 wt % unreacted fatty acid.
^{[2]} Commercially provided as a liquid containing 35 wt % of active surfactant compound.
^{[3]} Commercially provided as a solid powder containing about 100 wt % active ingredient.
^{[4]} 50% citric acid solution.
*The chelating agents were used in the following forms:
GLDA - tetrasodium salt
EDDS - trisodium salt
EDTA - free acid
MGDA - trisodium salt
ATMP - free phosphonic acid
EDTMP - free phosphonic acid

As will be known to a person skilled in the art, the components used as the sources of the active ingredients in Table 1 may include minor amounts of miscellaneous ingredients such as unreacted starting material and reaction by-products (i.e. to make each composition up to 100 wt %).

ATMP and EDTMP are phosphonate chelating agents (i.e. ATMP: Amino Trimethylene Phosphonic Acid; EDTMP: Ethylene Diamine Tetra(Methylene Phosphonic Acid).

All chelating agents were commercially provided as liquids containing around 33 to 38 wt % active.

The transmittance value was measured according to method 3 as described above and the yellowness index was measured according to method 1 as described above.

The yellowness index of the Blank sample and Composition 6 could not be determined on the Lovibond®2000+ Comparator due to their lack of transparency.

Results—Storage with Light Exposure

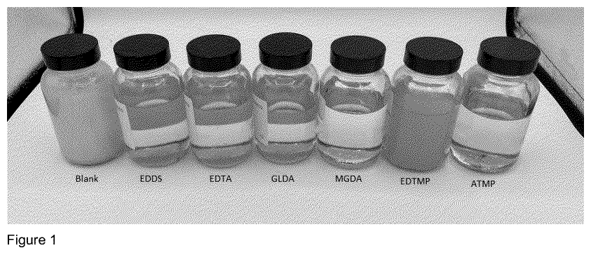

Compositions 1 to 6 were placed in closed transparent glass containers at ambient temperature and left by the window exposed to natural light. Light conditions were typical to standard UK summer light. The yellowness index and transmittance were measured after 4 weeks of light exposure, following the procedures described above. The results are recorded in Table 2 and displayed in FIG. 1.

TABLE 2

| Composition | 1 | 2 | 3 | 4 | 5 | 6 |
|---|---|---|---|---|---|---|
| Transmittance (%) | 100 | 100 | 100 | 100 | 100 | 0 |
| Yellowness index | 70 | 70 | 60 | 75 | 175 | N/A |
| (Hazen units) | | | | | | |

The results in Table 2 show that compositions 1 to 5 all remained transparent but that compositions 1 to 4 did not discolour/yellow, whereas composition 5 showed a significant level of discolouration/yellowing.

### Example 2

The following liquid compositions were prepared by mixing the components listed in Table 3 at a temperature between 4° and 55° C. All amounts are given as a percentage by weight (wt %) of the active ingredient. Compositions 8, 10, 12 and 14 are comparative compositions and do not contain an aminopolycarboxylic acid chelating agent.

TABLE 3

| | Amount of active ingredient (wt %) | | | | | | | |
|---|---|---|---|---|---|---|---|---|
| Composition | 7 | 8 | 9 | 10 | 11 | 12 | 13 | 14 |
| Cocamidopropyl | 6^{[5]} | 6^{[5]} | 6^{[6]} | 6^{[6]} | 0 | 0 | 6^{[7]} | 6^{[7]} |
| betaine | | | | | | | | |
| Lauramidopropyl | 0 | 0 | 0 | 0 | 6 | 6 | 0 | 0 |
| betaine^{[8]} | | | | | | | | |
| SCMI^{[1]} | 28.8 | 28.8 | 28.8 | 28.8 | 28.8 | 28.8 | 28.8 | 28.8 |
| GLDA^{[9]} | 1.2 | 0 | 1.2 | 0 | 1.2 | 0 | 1.2 | 0 |
| Sodium benzoate^{[3]} | 0.5 | 0.5 | 0.5 | 0.5 | 0.5 | 0.5 | 0.5 | 0.5 |
| Citric acid solution ^{[4]} | (q.s. to pH 6.5-6.9) | | | | | | | |
| DI water | 56.3 | 57.5 | 56.3 | 57.5 | 56.3 | 57.5 | 56.3 | 57.5 |

Components used as source of active ingredients:
^{[1]}SCMI was a commercially available source provided as solid flakes containing 80 wt % of active surfactant compound and containing 7 wt % unreacted fatty acid.
^{[3]}Commercially provided as a solid powder containing ~100 wt % active ingredient.
^{[4]} 50% citric acid solution.
^{[5]}C₁₂ fatty acid amidopropyl betaine. Commercially provided as a liquid containing 34 wt % active content.
^{[6]}Alternative C₁₂₋₁₈ fatty acid amidopropyl betaine. Commercially provided as a liquid containing 30 wt % active content.
^{[7]}C₈₋₁₈ fatty acid amidopropyl betaine. Commercially provided as a liquid containing 30 wt % active content.
^{[8]}Commercially provided as a liquid containing 30 wt % active content.
^{[9]}Commercially provided as a liquid containing 47 wt % active content and used as the tetrasodium salt.

As will be known to a person skilled in the art, the components used as the sources of the active ingredients in Table 3 may include minor amounts of miscellaneous ingredients such as unreacted starting material and reaction by-products (i.e. to make each composition up to 100 wt %).

The turbidity value was measured according to method 4 as described above and the yellowness index was measured according to method 2 as described above.

### Results—Storage in the Dark at 25° C.

Compositions 7 to 14 were placed in sealed transparent glass containers and stored in an oven at 25° C. for 12 weeks. The yellowness index and turbidity value were measured initially, after 8 weeks of storage in the dark, and after 12 weeks storage in the dark following methods 2 and 4 described above. The results are recorded in Table 4. A turbidity value of less than 10 NTU is considered clear.

TABLE 4

| Composition | 7 | 8 | 9 | 10 | 11 | 12 | 13 | 14 |
|---|---|---|---|---|---|---|---|---|
| Turbidity initial (NTU) | 0.84 | out of | 1.02 | out of | 1.19 | out of | 1.09 | out of |
| | | range | | range | | range | | range |
| Turbidity initial (visual | clear | hazy | clear | hazy | clear | hazy | clear | hazy |
| inspection) | | | | | | | | |
| Turbidity at 8 weeks | 1.37 | out of | 0.98 | out of | 0.88 | out of | 0.4 | out of |
| (NTU) | | range | | range | | range | | range |
| Turbidity at 8 weeks | clear | hazy | clear | hazy | clear | hazy | clear | hazy |
| (visual inspection) | | | | | | | | |
| Turbidity at 12 weeks | 1.46 | Out of | 1.12 | Out of | 1.09 | Out of | 0.86 | Out of |
| (NTU) | | range | | range | | range | | range |
| Turbidity at 12 weeks | Clear | Hazy | Clear | Hazy | Clear | Hazy | Clear | Hazy |
| (visual inspection) | | | | | | | | |
| Yellowness index initial | 63 | 420 | 75 | 447 | 66 | 407 | 59 | 485 |
| (Hazen units) | | | | | | | | |
| Yellowness index at 8 | 55 | 441 | 60 | 508 | 54 | 462 | 47 | 448 |
| weeks (Hazen units) | | | | | | | | |
| Yellowness index at 12 | 62 | 446 | 70 | 512 | 68 | 482 | 55 | 493 |
| weeks (Hazen units) | | | | | | | | |

“Out of range” is when a sample is too hazy or opaque for Turbidity to be measured.

The results in Table 4 show that the inventive compositions show low turbidity and low colour T initially and that this is maintained after 8 and 12 weeks of storage at 25° C.

### Results—Storage in the Dark at 5° C.

Compositions 7 to 14 were placed in closed transparent glass containers and stored in an oven at 5° C. for 12 weeks. The yellowness index and turbidity value were measured initially, after 8 weeks of storage in the dark and after 12 weeks storage in the dark, following methods 2 and 4 described above. The results are recorded in Table 5.

TABLE 5

| Composition | 7 | 8 | 9 | 10 | 11 | 12 | 13 | 14 |
|---|---|---|---|---|---|---|---|---|
| Turbidity initial (NTU) | 0.84 | out of | 1.02 | out of | 1.19 | out of | 1.09 | out of |
| | | range | | range | | range | | range |
| Turbidity initial | clear | hazy | clear | hazy | clear | hazy | clear | hazy |
| (visual inspection) | | | | | | | | |
| Turbidity at 8 weeks | 1.49 | out of | 1.92 | out of | 1.75 | out of | 1.59 | out of |
| (NTU) | | range | | range | | range | | range |
| Turbidity at 8 weeks | clear | hazy | clear | hazy | clear | hazy | clear | hazy |
| (visual inspection) | | | | | | | | |
| Turbidity at 12 weeks | 1.48 | Out of | 1.20 | Out of | 1.24 | Out of | 0.96 | Out of |
| (NTU) | | range | | range | | range | | range |
| Turbidity at 12 weeks | Clear | Hazy | Clear | Hazy | Clear | Hazy | Clear | Hazy |
| (visual inspection) | | | | | | | | |
| Yellowness index | 63 | 420 | 75 | 447 | 66 | 407 | 59 | 485 |
| initial (Hazen units) | | | | | | | | |
| Yellowness index at | 55 | 463 | 62 | 389 | 57 | 508 | 50 | 478 |
| 8 weeks (Hazen units) | | | | | | | | |
| Yellowness index at | 68 | 456 | 73 | 448 | 64 | 462 | 57 | 496 |
| 12 weeks (Hazen units) | | | | | | | | |

The results in Table 5 show that the inventive compositions show low turbidity and low colour initially and that this is maintained after 8 and 12 weeks of low temperature storage at 5° C.

### Results—Storage in the Dark at 45° C.

Inventive compositions 7, 9, 11 and 13 were placed in closed transparent glass containers and stored in an oven at 45° C. for 12 weeks. The yellowness index and turbidity value were measured initially, after 8 weeks of storage in the dark and after 12 weeks storage in the dark, following methods 2 and 4 described above. The results are recorded in Table 6.

TABLE 6

| Composition | 7 | 9 | 11 | 13 |
|---|---|---|---|---|
| Turbidity initial (NTU) | 0.84 | 1.02 | 1.19 | 1.09 |
| Turbidity initial (visual inspection) | clear | clear | clear | clear |
| Turbidity at 8 weeks (NTU) | 3.25 | 2.22 | 0.81 | 0.86 |
| Turbidity at 8 weeks (visual inspection) | clear | clear | clear | clear |
| Turbidity at 12 weeks (NTU) | 3.68 | 2.16 | 1.08 | 0.96 |
| Turbidity at 12 weeks (visual inspection) | Clear | Clear | Clear | Clear |
| Yellowness index initial (Hazen units) | 63 | 75 | 66 | 59 |
| Yellowness index at 8 weeks (Hazen units) | 74 | 71 | 56 | 62 |
| Yellowness index at 12 weeks (Hazen units) | 86 | 95 | 74 | 68 |

The results in Table 6 show that the inventive compositions show low turbidity and low colour initially and that this is maintained after 8 and 12 weeks of high temperature storage at 45° C.

Although a few preferred embodiments have been shown and described, it will be appreciated by those skilled in the art that various changes and modifications might be made without departing from the scope of the invention, as defined in the appended claims.

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention. Typically, when referring to compositions, a composition consisting essentially of a set of components will comprise less than 5% by weight, typically less than 3% by weight, more typically less than 1% by weight of non-specified components.

The term “consisting of” or “consists of” means including the components specified but excluding addition of other components.

Whenever appropriate, depending upon the context, the use of the term “comprises” or “comprising” may also be taken to encompass or include the meaning “consists essentially of” or “consisting essentially of”, and may also be taken to include the meaning “consists of” or “consisting of”.

As used herein, unless otherwise expressly specified, all numbers such as those expressing values, ranges, amounts of percentages may be read as if prefaced by the word “about”, even if the term does not expressly appear.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.70 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

For the avoidance of doubt, wherein amounts of components in a composition are described in wt %, this means the weight percentage of the specified component in relation to the whole composition referred to. For example, “wherein the surfactant composition comprises 1 to 20 wt % of amphoteric surfactant” means that from 1 to 20% of the total weight of the surfactant composition is provided by amphoteric surfactant.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. Use of a chelating agent in a surfactant composition to provide transparency and/or to substantially prevent discolouration, wherein the chelating agent comprises at least one aminopolycarboxylic acid or a salt thereof.

2. A method of providing transparency to and/or substantially preventing discolouration of a surfactant composition, the method comprising adding a chelating agent to the surfactant composition, wherein the chelating agent comprises at least one aminopolycarboxylic acid or a salt thereof.

3. The use according to claim 1, wherein the chelating agent provides transparency and substantially prevents discolouration.

4. The method according to claim 2, wherein the method provides transparency to and substantially prevents discolouration of the surfactant composition.

5. The method according to claim 2, wherein the surfactant composition comprises at least one surfactant independently selected from an anionic surfactant and an amphoteric surfactant, or mixtures thereof.

6. The method according to claim 5, wherein the anionic surfactant is an acyl alkyl isethionate surfactant of the formula (I): 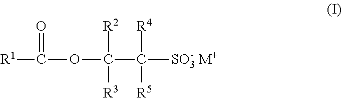
wherein R¹ represents an optionally substituted C₃-C₃₅ hydrocarbyl group; each of R², R³, R⁴ and R⁵ independently represents hydrogen or a C₁-C₄ alkyl group and wherein at least one of R², R³, R⁴ and R⁵ is not hydrogen; and M⁺ represents a cation.

7. The method according to claim 5, wherein the amphoteric surfactant is a betaine.

8. The method according to claim 5, wherein the surfactant composition comprises:
(i) at least one acyl alkyl isethionate surfactant of the formula (I): 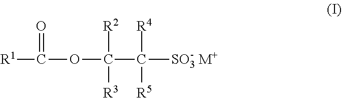
wherein R¹ represents an optionally substituted C₃-C₃₅ hydrocarbyl group; each of R², R³, R⁴ and R⁵ independently represents hydrogen or a C₁-C₄ alkyl group and wherein at least one of R², R³, R⁴ and R⁵ is not hydrogen; and M⁺ represents a cation; and
(ii) at least one amphoteric surfactant, for example at least one betaine surfactant.

9. The method according to claim 2, wherein the chelating agent provides a transparent surfactant composition without substantial discolouration upon storage for a period of at least 2 weeks, for example at a temperature above ambient temperature and/or upon exposure to light.

10. The method according to claim 2, wherein the chelating agent provides a transparent surfactant composition having a transmittance value of 90% or greater and/or a yellowness index of 125 Hazen units or less upon storage for a period of at least 2 weeks, for example at a temperature above ambient temperature and/or upon exposure to light.

11. A transparent surfactant composition comprising:
(i) from 15 to 98 wt % (for example from 15 to 95 wt %) of at least one acyl alkyl isethionate surfactant of the formula (I): 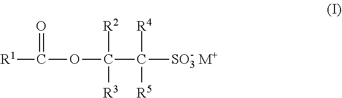
wherein R¹ represents an optionally substituted C₃-C₃₅ hydrocarbyl group; each of R², R³, R⁴ and R⁵ independently represents hydrogen or a C₁-C₄ alkyl group and wherein at least one of R², R³, R⁴ and R⁵ is not hydrogen; and M⁺ represents a cation;
(ii) from 3 to 12 wt % of at least one amphoteric surfactant; and
(iii) from 0.1 to 10 wt % of at least one chelating agent, wherein the chelating agent comprises at least one aminopolycarboxylic acid or a salt thereof.

12. The transparent surfactant composition according to claim 11, wherein the composition remains transparent without substantial discolouration upon storage for a period of time, such as at least 2 weeks.

13. The transparent surfactant composition according to claim 11, wherein the composition remains transparent without substantial discolouration upon storage for a period of at least 2 weeks at a temperature above ambient temperature and/or upon exposure to light.

14. The transparent surfactant composition according to claim 11, wherein the composition has a transmittance value of 90% or greater and/or a yellowness index of 125 Hazen units or less upon storage for a period of at least 2 weeks, for example at a temperature above ambient temperature and/or upon exposure to light.

15. The composition according to claim 11, wherein the at least one chelating agent is independently selected from ethylenediamine-N,N′-diglutaric acid (EDDG), ethylenediamine-N,N′-dimalonic acid (EDDM), iminodisuccinic acid (IDS), ethylenediaminetetraacetic acid (EDTA), ethylenediaminedisuccinic acid (EDDS), methylglycinediacetic acid (MGDA), L-glutamic acid-N,N-diacetic acid (GLDA), diethylene triamine pentaacetic acid (DTPA), L-aspartic acid diacetic acid (ASDA), hydroxyethyl ethylenediaminetriacetic acid (HEDTA), iminodifumaric acid (IDF), iminoditartaric acid (IDT), iminodimaleic acid (IDMAL), iminodimalic acid (IDM), ethylenediaminedifumaric acid (EDDF), ethylenediaminedimalic acid (EDDM), ethylenediamineditartaric acid (EDDT) and ethylenediaminedimaleic acid (EDDMAL), preferably from EDDG, EDDM, IDS, EDTA, EDDS, MGDA and GLDA, more preferably from EDTA, EDDS, MGDA and GLDA, or a salt thereof.

16. The composition according to claim 11, wherein the chelating agent is GLDA, or a salt thereof.

17. A personal or household cleaning composition comprising a transparent surfactant composition according to claim 11.

18. A method of preparing a transparent surfactant composition according to claim 11, the method comprising admixing the at least one acyl alkyl isethionate of the formula (I), the at least one betaine surfactant and the at least one chelating agent in the amounts set out in claim 11.
